(19) United States
(12) Patent Application Publication
(10) Pub. No.: US 2003/0180290 A1
(43) Pub. Date: Sep. 25, 2003

(54) Anti-CD80 antibody having ADCC activity for ADCC mediated killing of B cell lymphoma cells alone or in combination with other therapies

(75) Inventors: Kandasamy Hariharan, San Diego, CA (US); Nabil Hanna, Rancho Santa Fe, CA (US)

Correspondence Address: Pillsbury Winthrop LLP, Intellectual Property Group, P.O. Box 10500, McLean, VA, 22102, US

(73) Assignee: IDEC Pharmaceuticals Corporation, San Diego, CA

(21) Appl. No.: 10/291,532

(22) Filed: Nov. 12, 2002

Related U.S. Application Data
(63) Continuation-in-part of application No. 09/758,173, filed on Jan. 12, 2001.
(62) Division of application No. 09/383,916, filed on Aug. 26, 1999.
(62) Division of application No. 08/487,950, filed on Jun. 7, 1995, now Pat. No. 5,612,045.
(60) Provisional application No. 60/331,187, filed on Nov. 9, 2001.

(51) Int. Cl. A61K 39/395

(52) U.S. Cl. 424/132.1; 424/144.1; 424/141.1

(57) ABSTRACT

Methods for treating B cell malignancies, in particular B cell leukemia and lymphoma, using an anti-CD80 antibody alone or in combination with an anti-CD20 antibody or chemotherapy is provided. These methods result in a synergistic anti-tumor response.

### CROSS REFERENCE TO RELATED APPLICATIONS

This application claims priority to provisional application U.S. Serial No. 60/331,187 filed Nov. 9, 2001 which is incorporated by reference in its entirety herein. Additionally, this application is a continuation-in-part of U.S. Ser. No. 09/758,173 filed Jan. 12, 2001 which is a divisional of U.S. Ser. No. 09/383,916 filed Aug. 26, 1999 which is a divisional of U.S. Ser. No. 08/487,950 filed Jun. 7, 1995, now U.S. Pat. No. 6,113,898 all of which are incorporated by reference in their entirety herein.

### FIELD OF THE INVENTION

The present invention relates to the discovery that a PRIMATIZED® IgG₁ antibody that shows specificity to the human CD80 molecule and which is referred to by the subject assignee, IDEC Pharmaceuticals Corporation, as IDEC-114 possesses antibody dependent cellular cytotoxicty (ADCC) against CD80 positive cells, especially CD80 positive cells of B cell lineage, and more particularly B cell lymphoma cells. (The sequence of this PRIMATIZED® antibody is disclosed in U.S. Pat. No. 6,113,898 which is incorporated by reference in its entirety herein). (This primatized antibody is referred to as 16C10 therein). The present invention also relates to the discovery that the use of IDEC-114 in combination with Rituxan®, a chimeric anti-CD20 antibody approved by the FDA for treatment of non-Hodgkin's lymphoma, and/or chemotherapy yields a synergistic anti-tumor response against B cell lymphoma in vivo.

### BACKGROUND OF THE INVENTION

IDEC-114 or 16C10 as it is referred to in an earlier patent and applications by the inventors is a PRIMATIZED® anti-CD80 IgG₁ lambda monoclonal antibody (mAb) containing human constant regions and primate (cynomolgus macaque) variable regions. This antibody binds specifically to human CD80 (B7.1), which is membrane-associated 60 KDa glycoprotein expressed an activated B cells, activated antigen presenting cells, and activated T cells. (Freeman et al., J. Immunol. 1043:2714-22 (1989); Razi-Wolf et al., Proc. Natl Acad. Sci., USA 89:4210-4 (1992); and Azuma et al., J. Exp. Med. 177:845-50 (1993)) As noted above, the DNA and amino acid sequences that contain the variable heavy and light regions of IDEC-114 are disclosed in U.S. Pat. No. 6,113,898, which are identified therein as the 16C10 and heavy and light variable sequences, and which patent is incorporated by reference in its entirety herein.

Much has been learned about the function of CD80 and their family members as costimulatory molecules for eliciting T-cell responses (Freeman et al., (Id) and Hatchcock et al., J. Exp. Med. 180(2): 631-40 (1999)). Various strategies, including antibody-based approaches aimed at blocking the interaction between CD80 and its ligand on T cells (CD28, CTLA-4), have been pursued as a treatment modality for autoimmune diseases and transplantation. (Anderson et al., Curr. Opn. Immunol. 11(6): 677-83 (1999)). In addition to activated B cells, CD80 is expressed on malignant B cells. (Freeman et al., (Id) and Durfma et al., Blood 90(11): 4297-4309 (1997)).

Rituxmab (Rituxan®) is an unconjugated chimeric IgG₁ mAb that shows specificity to the human leukocyte antigen CD20. This antibody was developed at IDEC Pharmaceuticals Corporation and was approved for use by the Food and Drug Administration for treatment of relapsed or refractory, low-grade or follicular B-cell non-Hodgkin's lymphoma. (Rituxan® Package Insert San Diego: IDEC Pharm. Corp., 4-19, 2001, 2). The DNA and amino acid sequence for Rituxan® are disclosed in U.S. Pat. No. 5,736,137, which patent is incorporated by reference in its entirety herein. Additionally, a CHO cell transfectoma TCAE8 that expresses Rituxan® has been deposited with the American Type Culture Collection (ATCC) and accorded ATCC deposit number 69119.

### SUMMARY OF THE INVENTION

IDEC-114 is a PRIMATIZED® antibody that shows specificity to the human CD80 molecule. CD80 is expressed on activated B cells and on B-cell lymphoma. In earlier applications, referred to above, it was described that the use of anti-CD80 antibodies for treatment of lymphoma was within the scope of the invention disclosed therein. This invention provides further information on this specific usage of anti-CD80 antibodies, and more particularly the primatized anti-CD80 antibodies which are the subject of U.S. Pat. No. 6,113,898 and the divisional applications, thereof, which are incorporated by reference herein. Particularly, the present inventors further evaluated whether IDEC-114 alone or in combination with rituxmab (Rituxan®) exhibits an antitumor response against B-cell lymphoma in different experimental systems. These in vitro cytotoxicty studies showed that IDEC-114, like rituxmab, kills CD80⁺ SB and SKW lymphoma cells via Fc-dependent host effector cell-mediated cytotoxicity (ADCC). Combination studies using saturating concentrations of IDEC-114 and sub-optimal concentrations of rituxmab resulted in enhanced ADCC. IDEC-114 also exhibited complement-dependent cytotoxicity (CDC) with CD80 high expressing CHO cell transfectants, but failed to mediate CDC with CD80⁺ B-lymphoma cell lines expressing much lower levels of antigen. These findings indicate that Fc-mediated effector mechanisms of IDEC-114 are more sensitive than complement when target cells contain limited amounts of antigen. Furthermore, in vivo testing of IDEC-114 in a human B lymphoma/SCID mouse model demonstrated antitumor activity at 100, 200, and 400 μg per injection. The antitumor response observed with IDEC-114 was comparable to the antitumor response observed with rituximab at the same dose and treatment schedule. Using the same dosing schedule, the combination of IDEC-114 with rituximab produced synergistic antitumor activity compared with either antibody alone. Mice injected with 200 μg of IDEC-114 and 200 μg of rituximab showed a significantly higher disease-free survival compared with mice injected with either 200 μg of IDEC-114 (p<0.005) or 200 μg of rituximab (p<0.001). Furthermore, a significantly greater antitumor response was observed in mice that received the IDEC-114/rituximab combination therapy compared with that observed in mice that received 400 μg of IDEC-114 (p<0.001) or 400 μg of rituximab (p<0.001) at the same dosing schedule. Overall the results of this study indicate that the combination of IDEC-114 with rituximab can provide a synergistic antitumor response against B-cell lymphoma in vivo.

### OBJECTS OF THE INVENTION

It is a specific object of the invention to use a PRIMATIZED® anti-CD80 antibody, IDEC-114, for mediating ADCC of CD80 positive cells, especially cells of the B cell lineage.

It is more specific object of the invention to use IDEC-114 or another anti-CD80 antibody for treatment of B cell lymphoma or other B cell malignancies.

It is another specific object of the invention to use IDEC-114 in combination with an anti-CD20 antibody, preferably RITUXAN®, for treatment of B cell lymphoma or other B cell malignancies.

It is another specific object of the invention to use IDEC-114 or another anti-CD80 antibody in combination with a chemotherapeutic agent for treatment of B cell lymphoma or other B cell malignancies.

It is an object of the invention to use an anti-CD20 antibody, preferably RITUXAN®, to potentiate the ADCC activity of an anti-CD80 antibody, preferably IDEC-114.

### DETAILED DESCRIPTION OF THE FIGURES

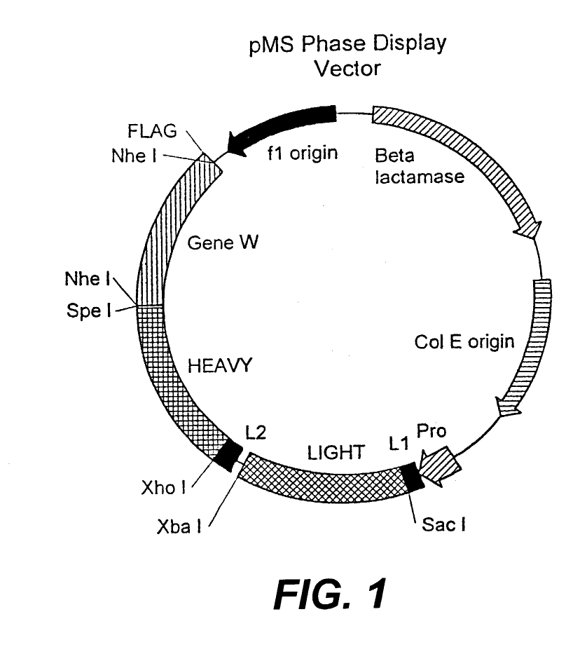

FIG. 1 depicts the pMS vector used to screen recombinant immunoglobulin libraries produced against B7 displayed on the surface of filamentous phage which contains primers based on macaque immunoglobulin sequences.

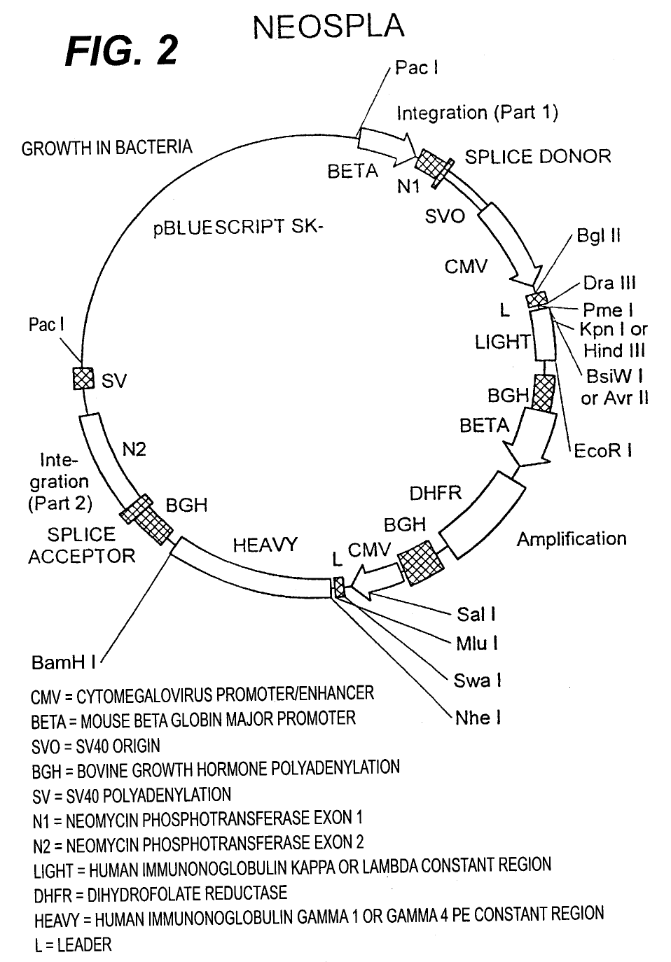

FIG. 2 depicts the NEOSPLA expression vector used to express the subject primatized antibodies specific to human B7.1 antigen.

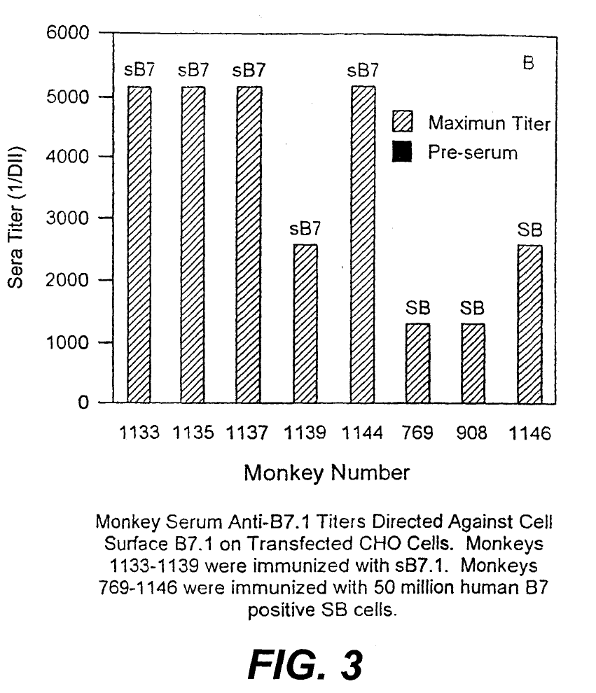

FIG. 3 depicts monkey serum anti-B7.1 titers directed against cell surface B7.1 on transfected CHO cells.

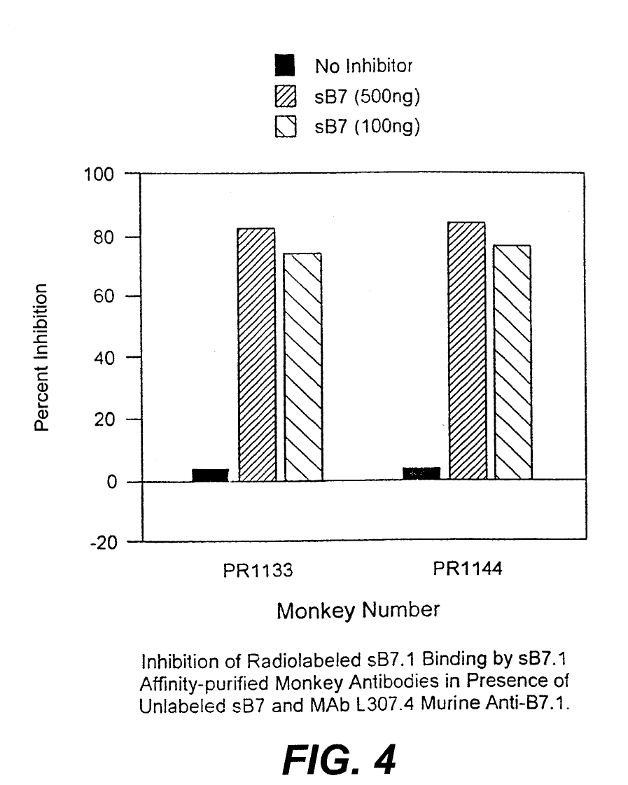

FIG. 4 depicts inhibition of radiolabeled sB7.1 binding by SB7.1 affinity-purified monkey antibodies in the presence of unlabeled SB7 and Mab L307.4 murine anti-B7.1.

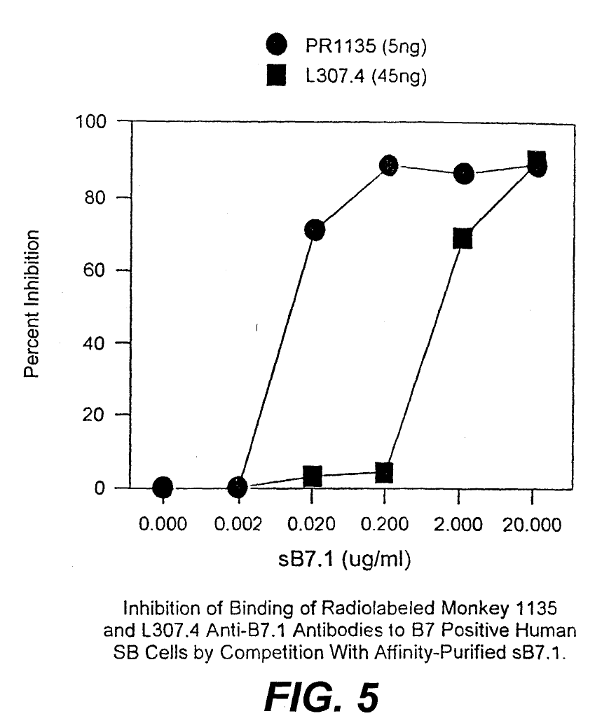

FIG. 5 depicts inhibition of binding of radiolabeled monkey 135 and L3707.4 anti-B7.1 antibodies to B7 positive human SB cells by competition with affinity-purified SB7.1.

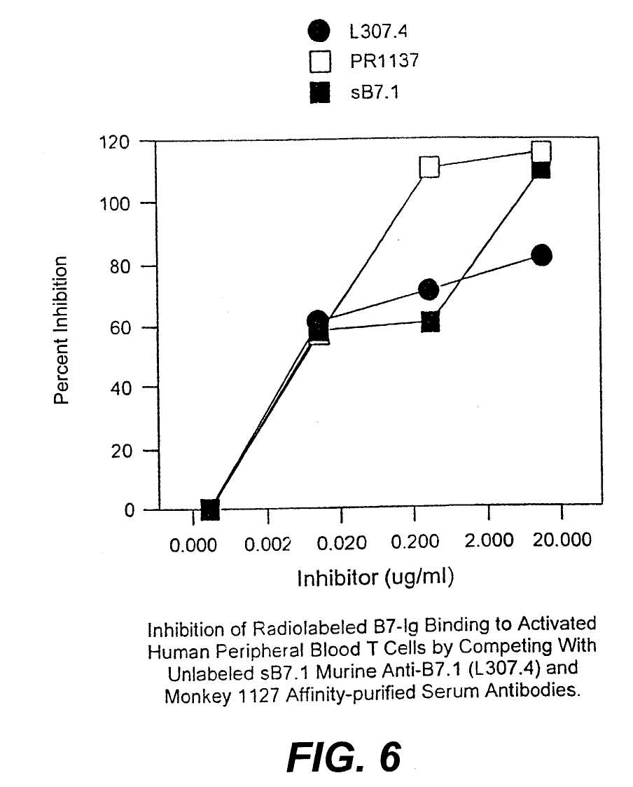

FIG. 6 depicts inhibition of radiolabeled B7-Ig binding to activated human peripheral blood T cells by competing with unlabeled SB7.1 murine anti-B7.1 (L307.4) and monkey 1127 affinity purified serum antibodies.

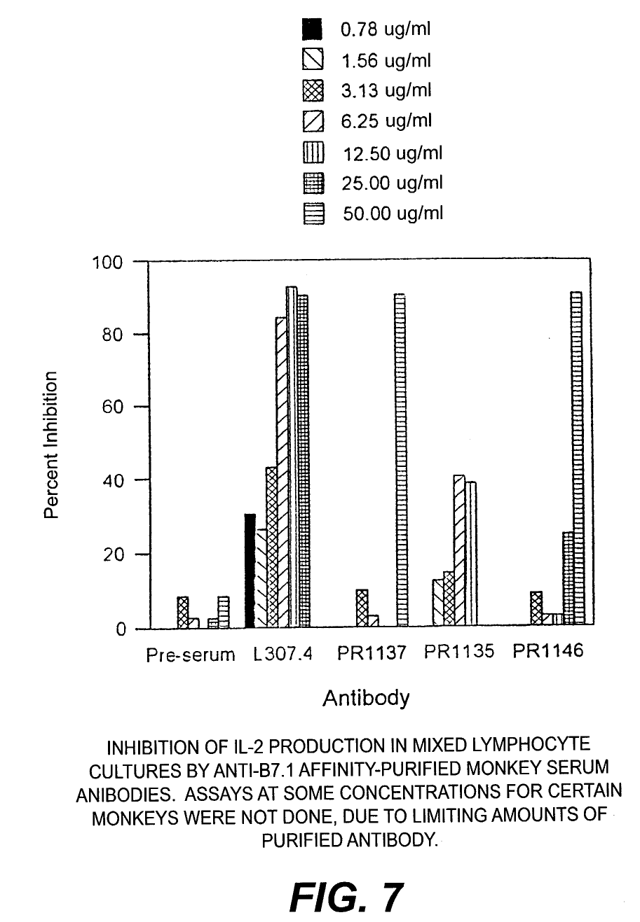

FIG. 7 depicts inhibition of IL-2 protein in mixed lymphocyte cultures by anti-B7.1 affinity-purified monkey serum antibodies.

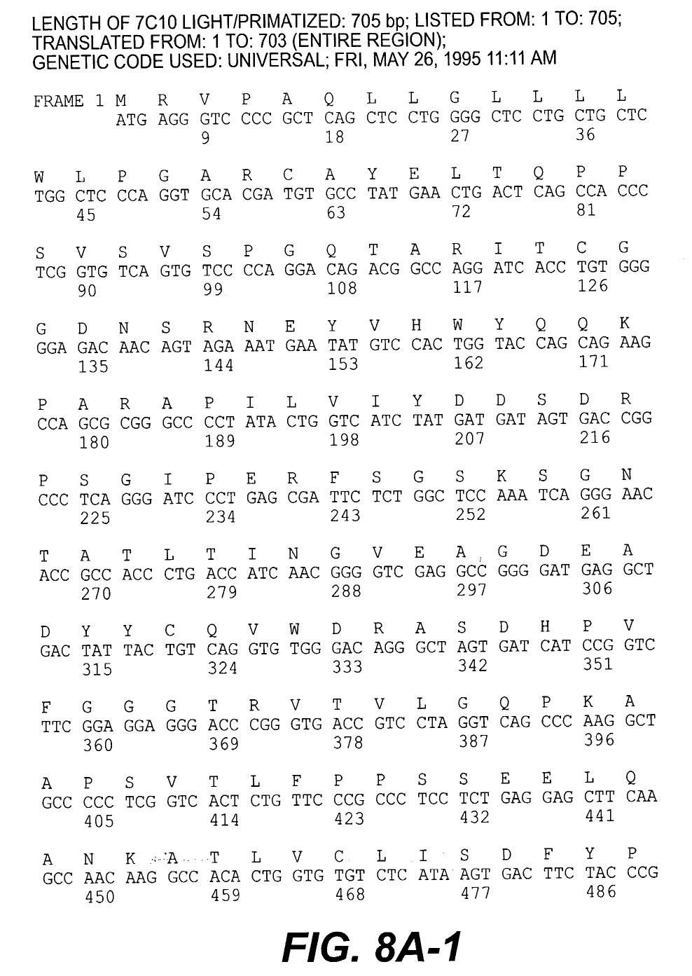

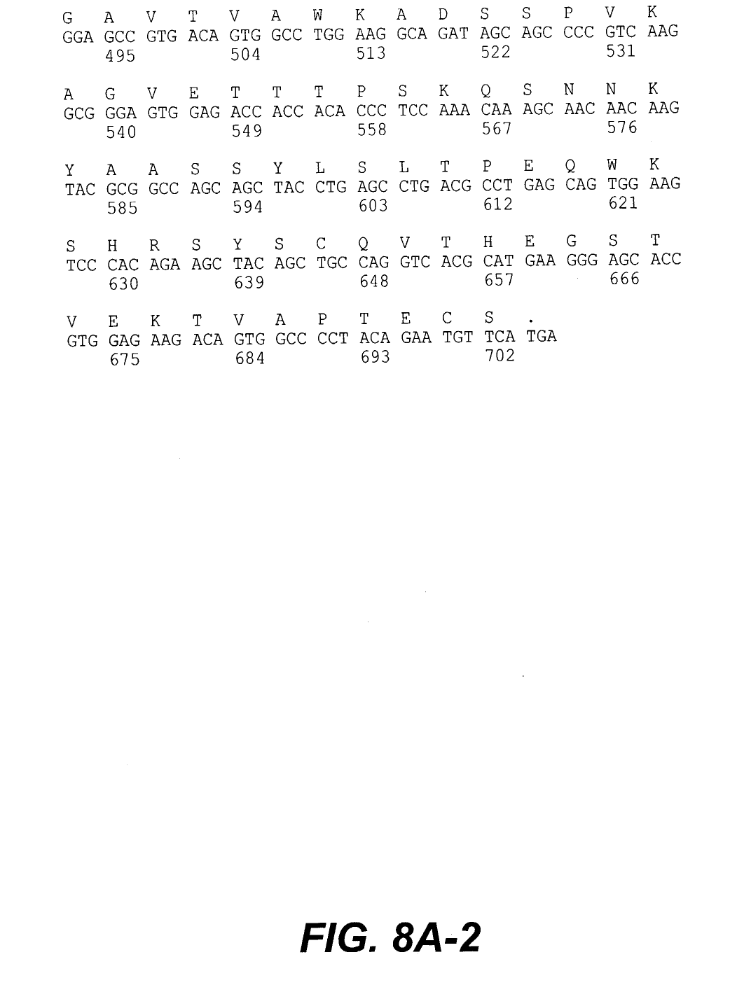

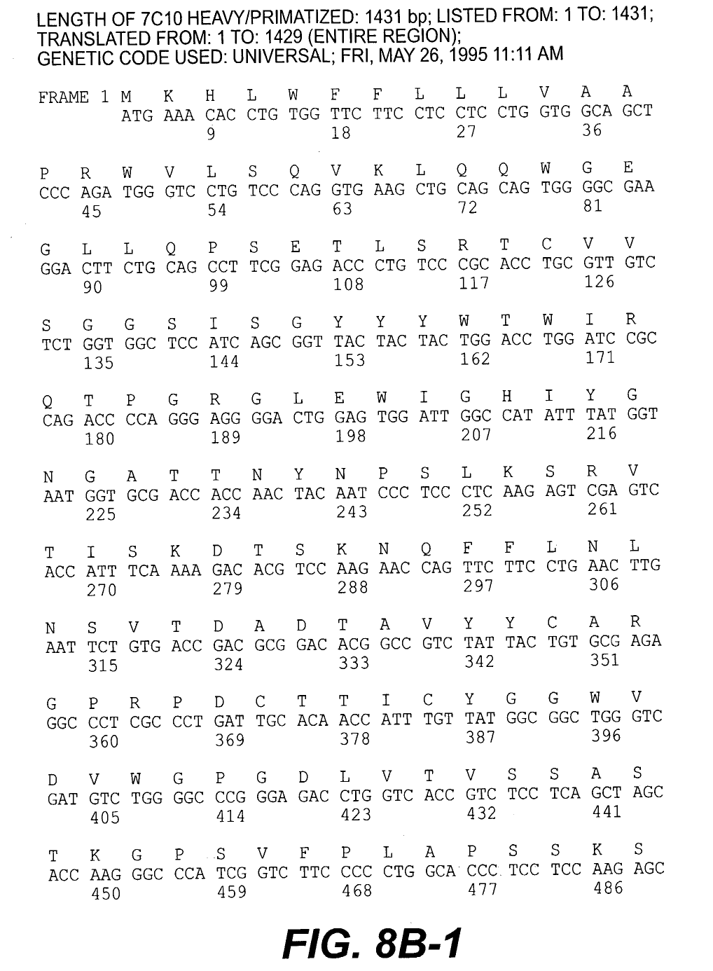

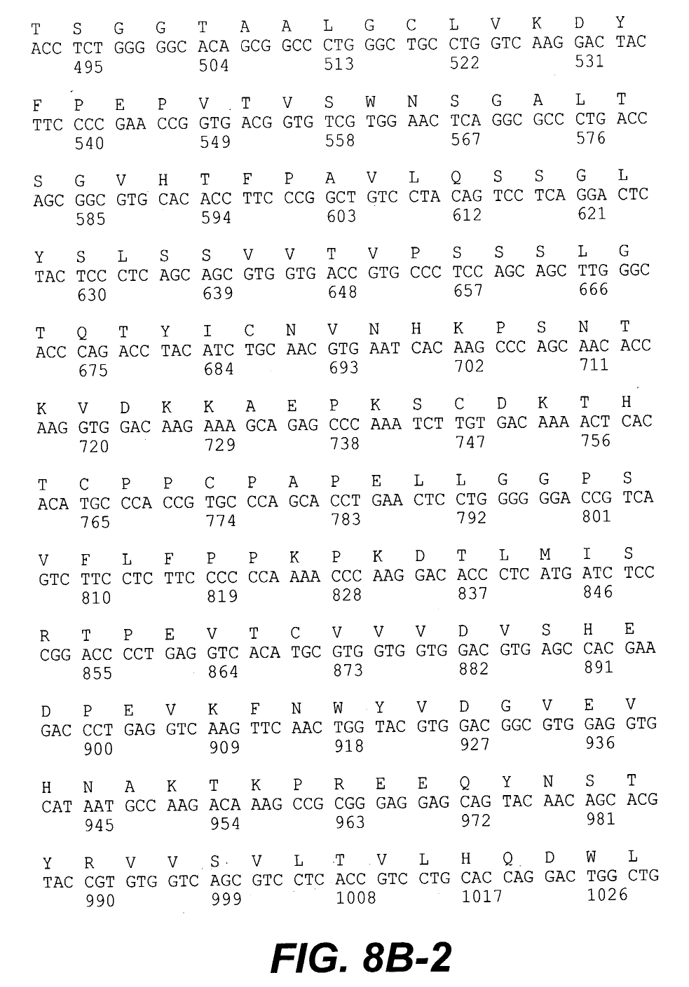

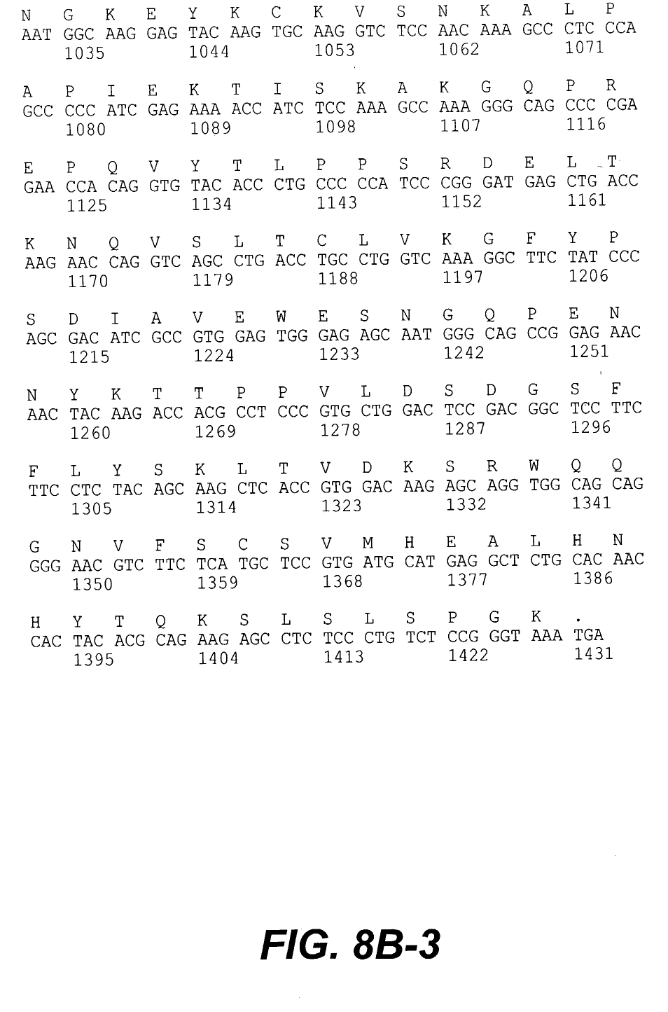

FIG. 8a depicts the amino acid and nucleic acid sequence of a primatized form of the light chain of 7C10.

FIG. 8b depicts the amino acid and nucleic acid sequence of a primatized form of the heavy chain of 7C10.

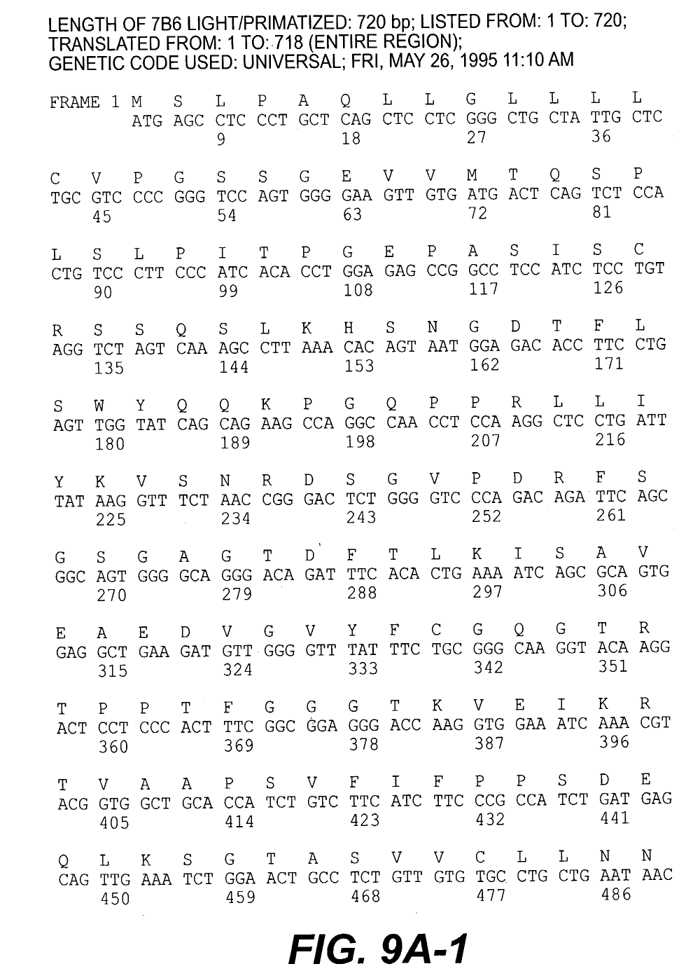

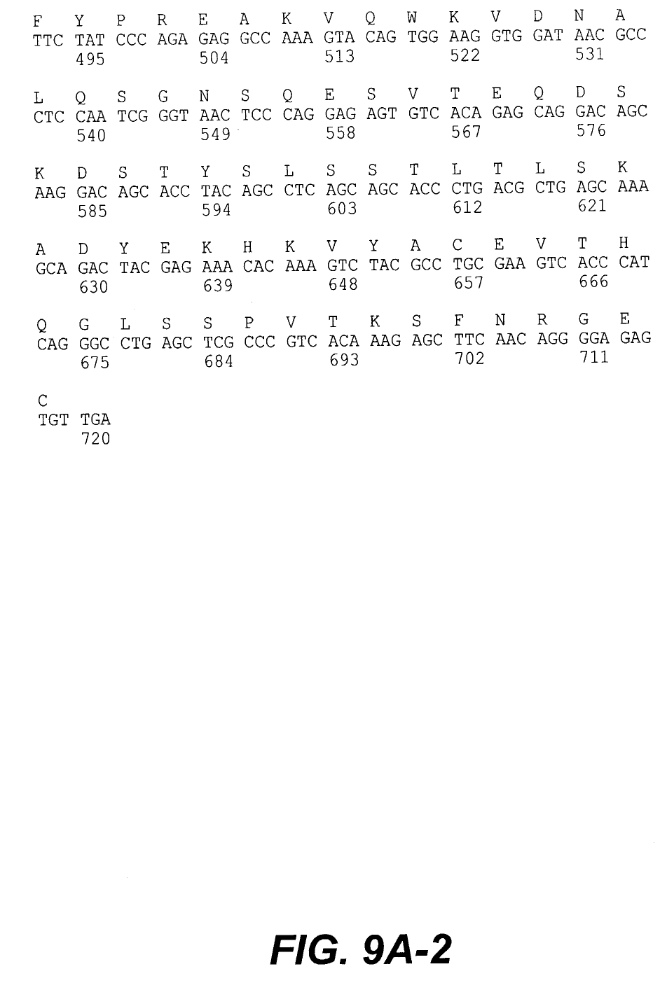

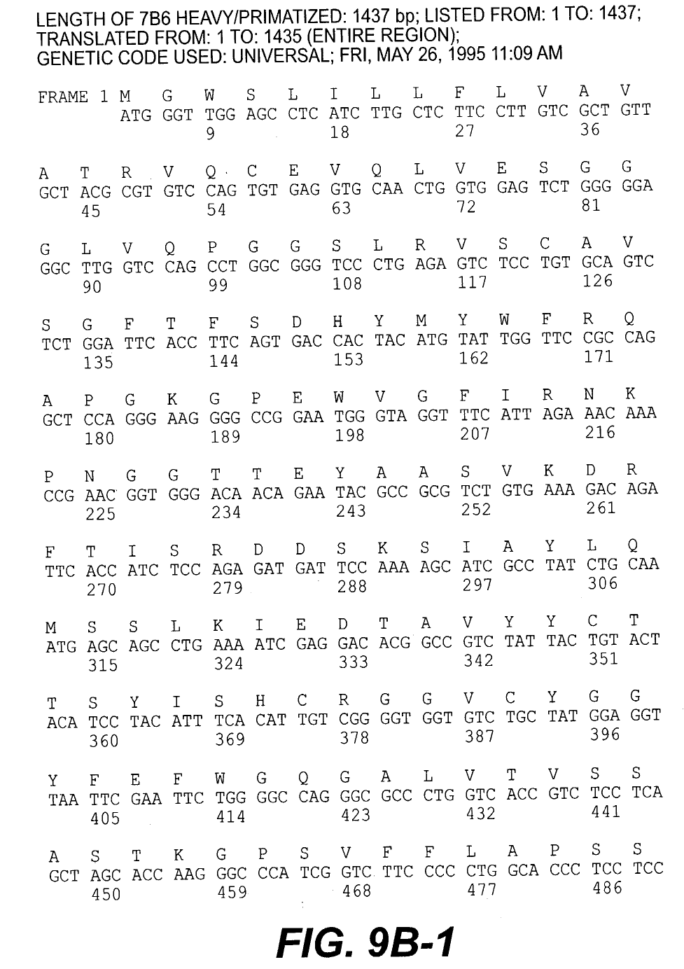

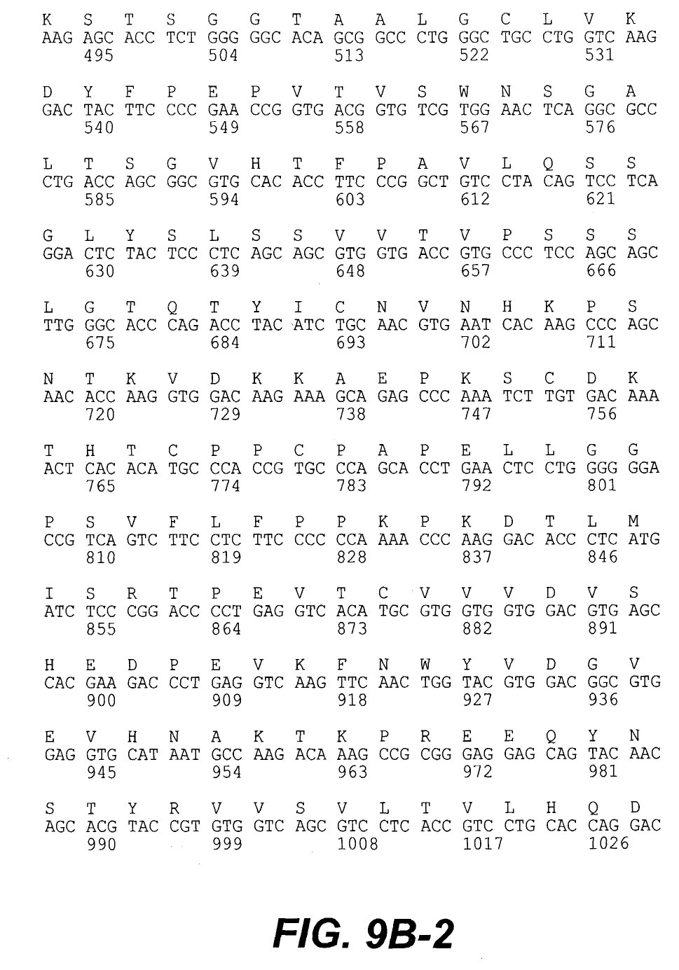

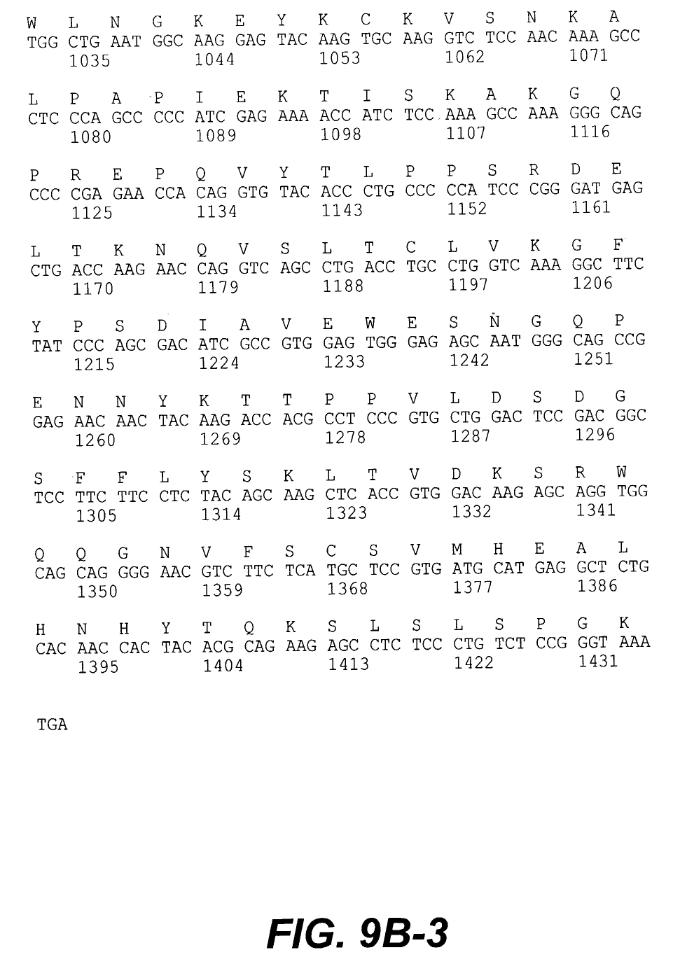

FIG. 9a depicts the amino acid and nucleic acid sequence of a primatized form of the light chain of 7B6.

FIG. 9b depicts the amino acid and nucleic acid sequence of a primatized form of the heavy chain of 7B6.

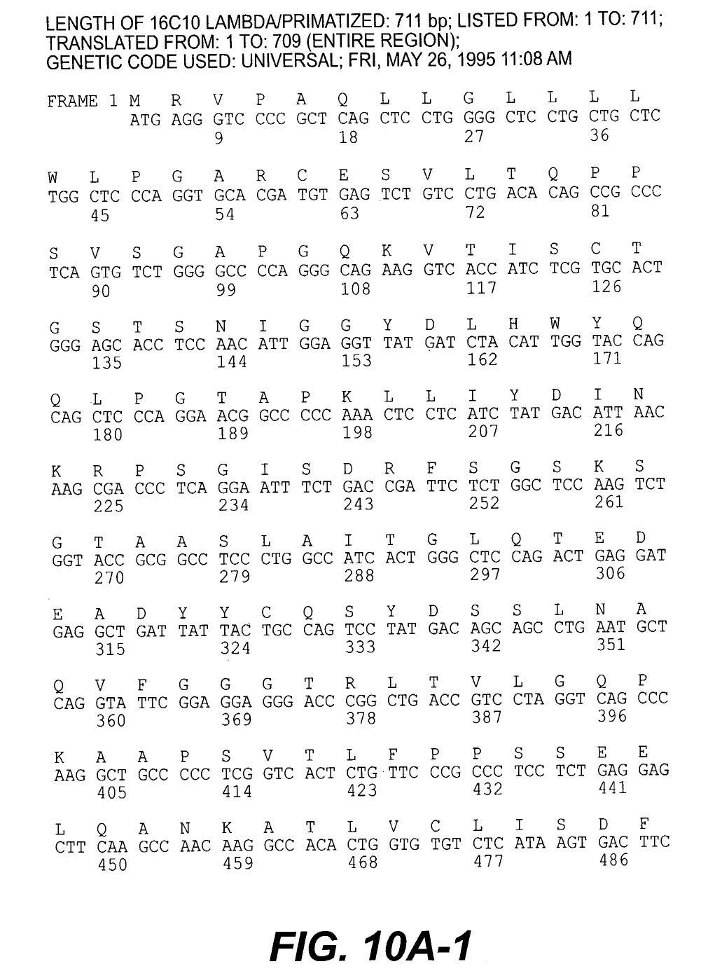

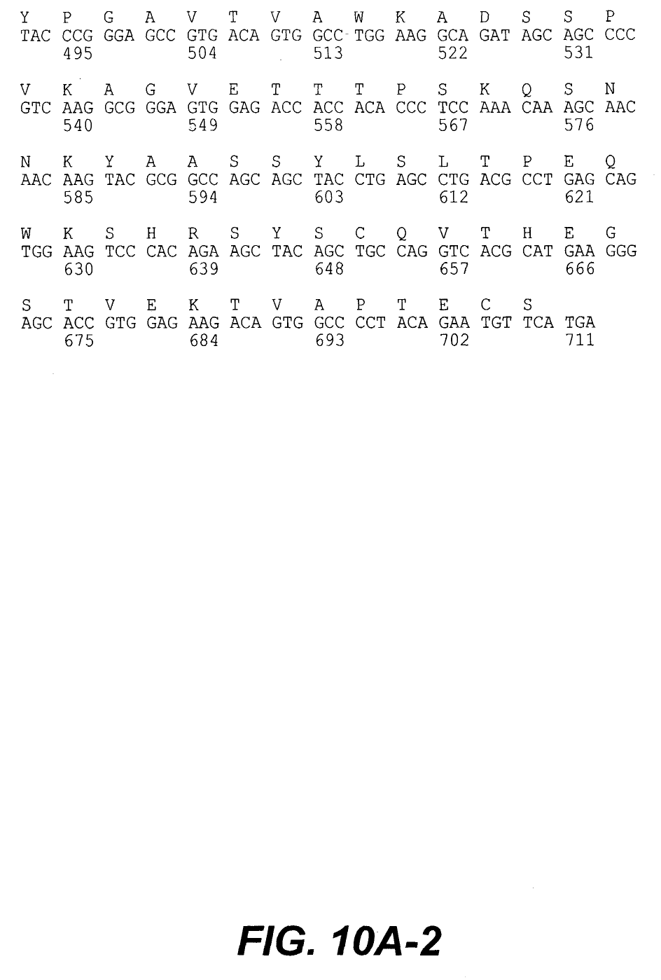

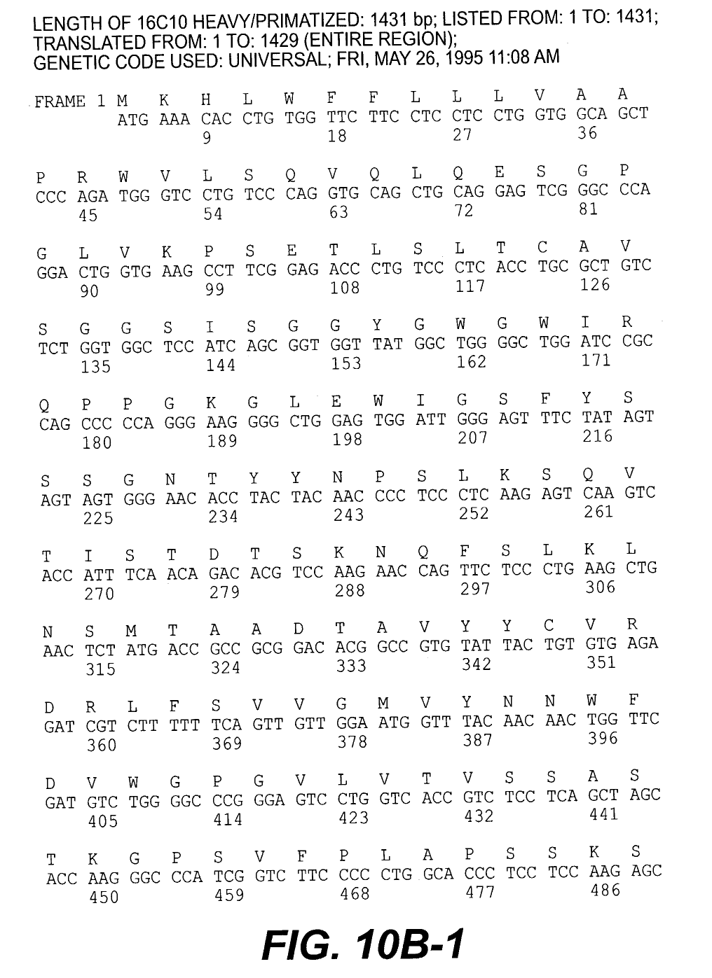

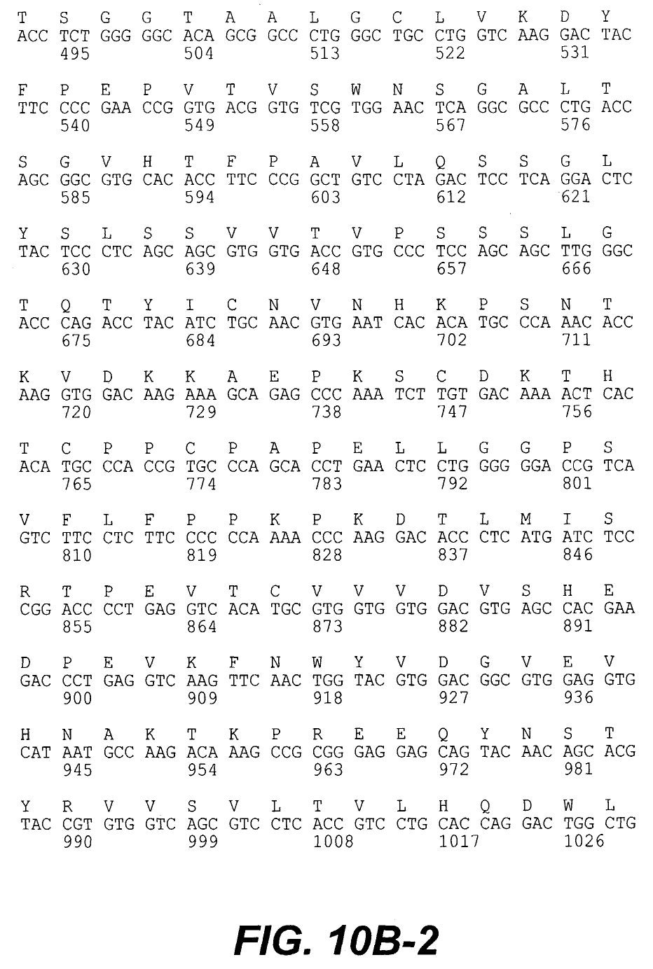

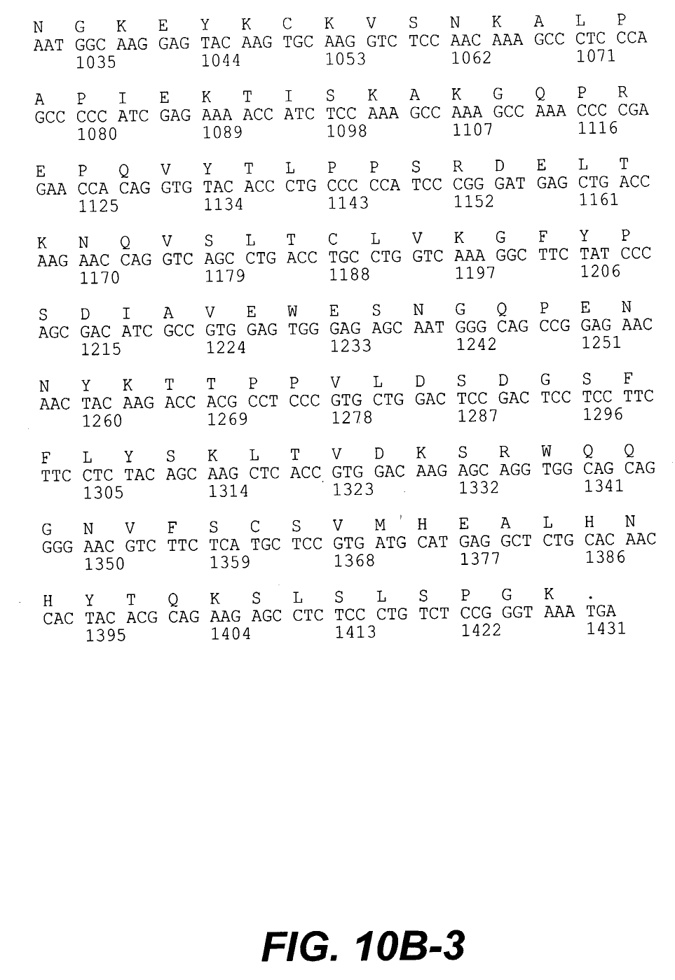

FIG. 10a depicts the amino acid and nucleic acid sequence of a primatized light chain 16C10.

FIG. 10b depicts the amino acid and nucleic acid sequence of a primatized heavy chain 16C10.

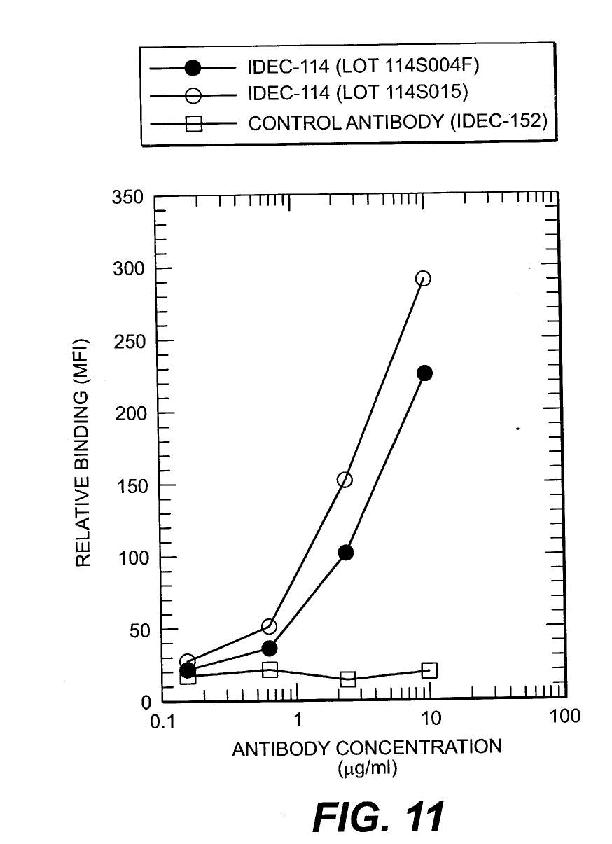

FIG. 11 compares the binding activity of two different lots of IDEC-114 to membrane bound CD80 cells determined by flow cytometry using CHO cells which express the CD80 molecule.

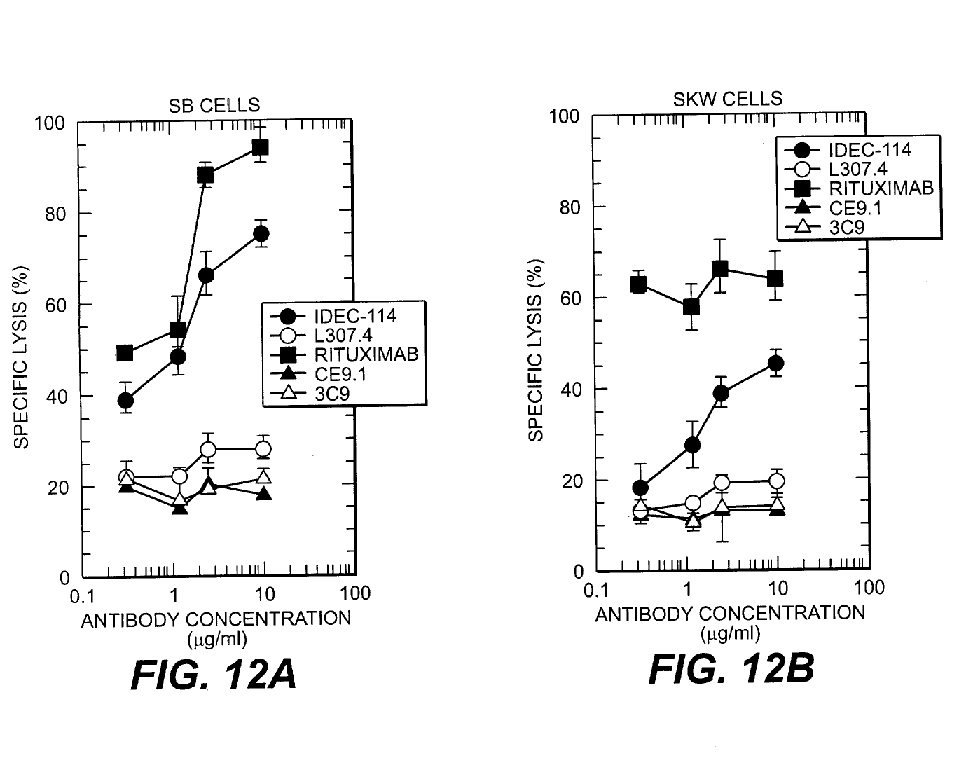

FIG. 12 shows the ADCC activity of IDEC-114 and RITUXAN® against SB cells and SKW cells by methods described in detail infra. ⁵¹Cr-labeled SB or SKW cells were incubated with varying concentration of IDEC-114, RITUXAN®, human CE9-1 (an irrelevant isotype-matched control anti-human CD4 antibody) or murine (3C9) (an irrelevant isotype-matched control antibody) on activated human effector cells from peripheral blood at a 50:1 effector to target ratio. ⁵¹Cr released from target cells was measured and the percentage of specific lysis was then determined.

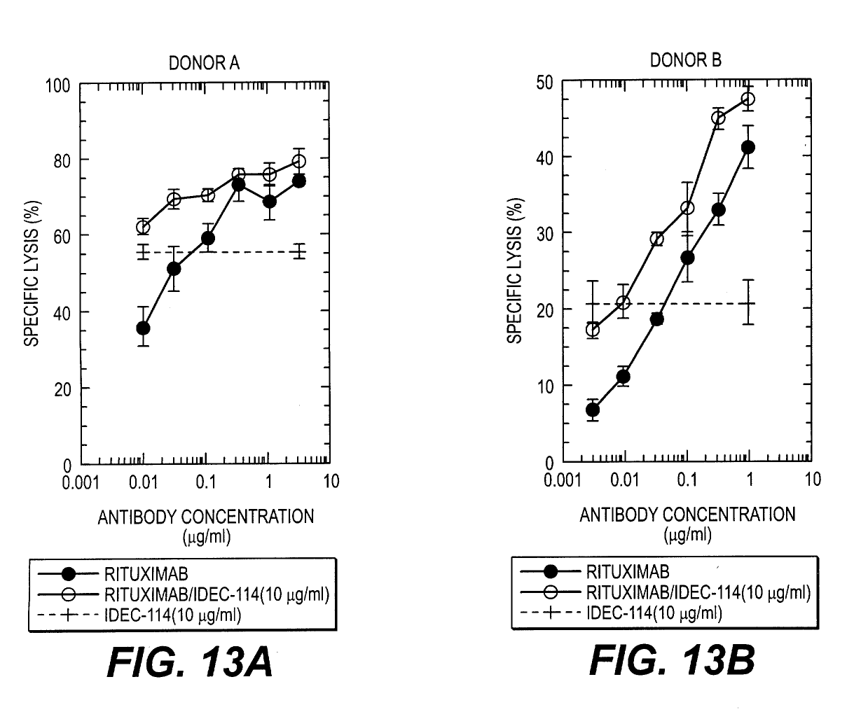

FIG. 13 shows the ADCC activity of IDEC-114 and rituximab (RITUXAN®) combination. The ADCC activity of IDEC-114 and rituximab in combination was determined on SKW cells as described infra. (Antibody-Dependent Cellular Cytotoxicity [ADCC]). ⁵¹Cr-labeled SKW cells were incubated with 10 μg/ml of IDEC-114 rituximab and activated host effector cells from peripheral blood of two donors (A and B) at 50:1 effector to target ratio. ⁵¹Cr released from the target cells was measured and the percentage of specific lysis was determined.

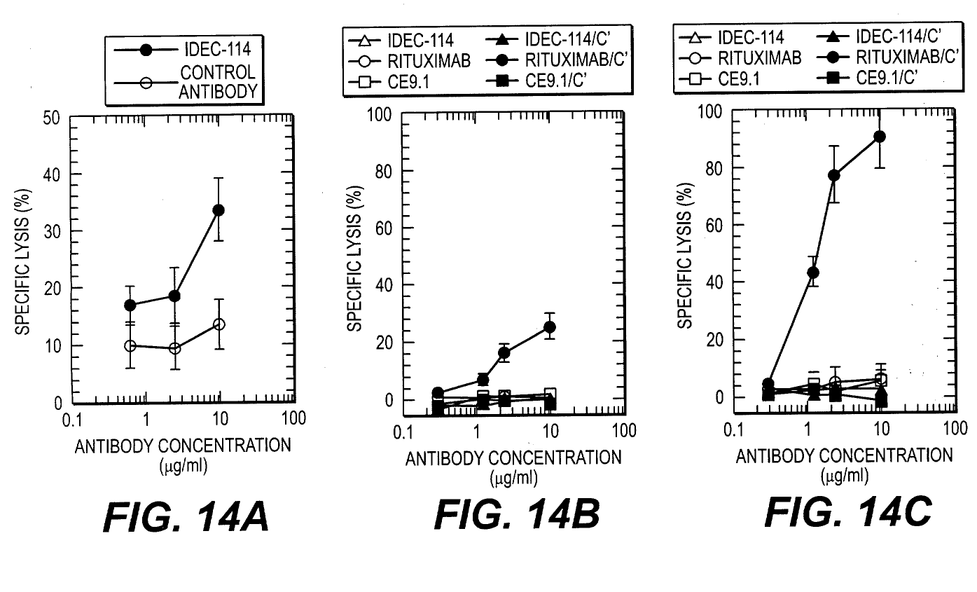

FIG. 14 shows the CDC activity of IDEC-114. The CDC activity of IDEC-114 and rituximab was determined on CD80-expressing CHO (a), SKW (b), or Daudi (c) ells as described infra in the examples. (Complement-Dependent Cytotoxicity [CDC]). ⁵¹Cr-labeled target cells were incubated with IDEC-114, rituximab, or control antibodies at indicated antibody concentrations with and without complement. After 4 hours of incubation, ⁵¹Cr released from the target cells was measured and the percentage of specific lysis was determined.

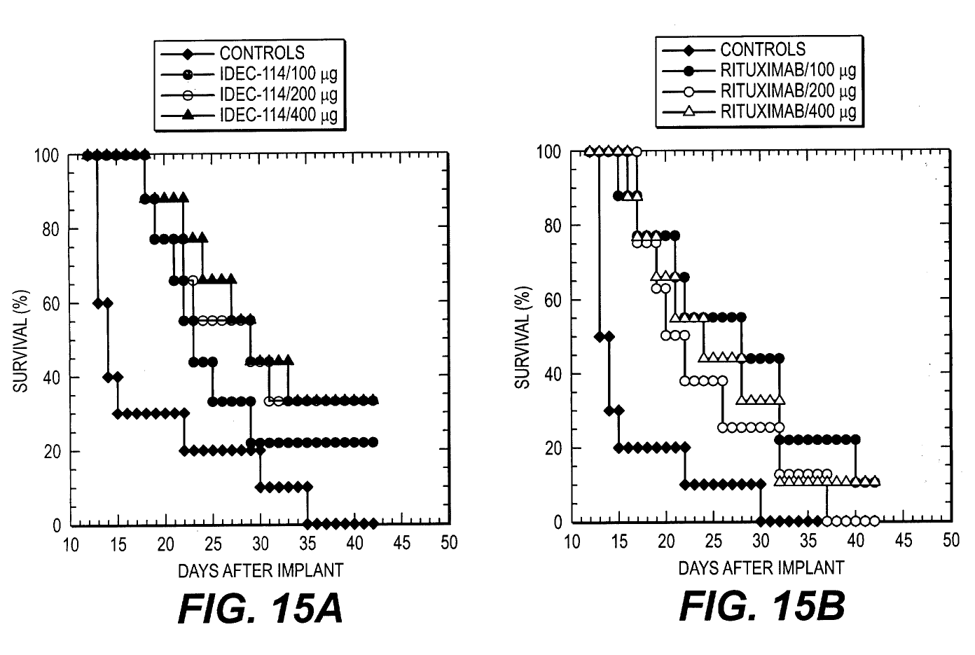

FIG. 15 shows the antitumor response elicited by IDEC-114 in SKW/SCID mice. On day 0, groups of mice were inoculated intravenously with 3×10⁶ SKW cells. Mice in the treatment groups (N=8) were injected intraperitonealy with IDEC-114 (a) or rituximab (b) at 100 μg, 200 μg, or 400 μg on days 1, 3, 5, 7, 9, and 11. All antibody injections were given in a final volume of 200 μl. Mice were monitored for disease development and death.

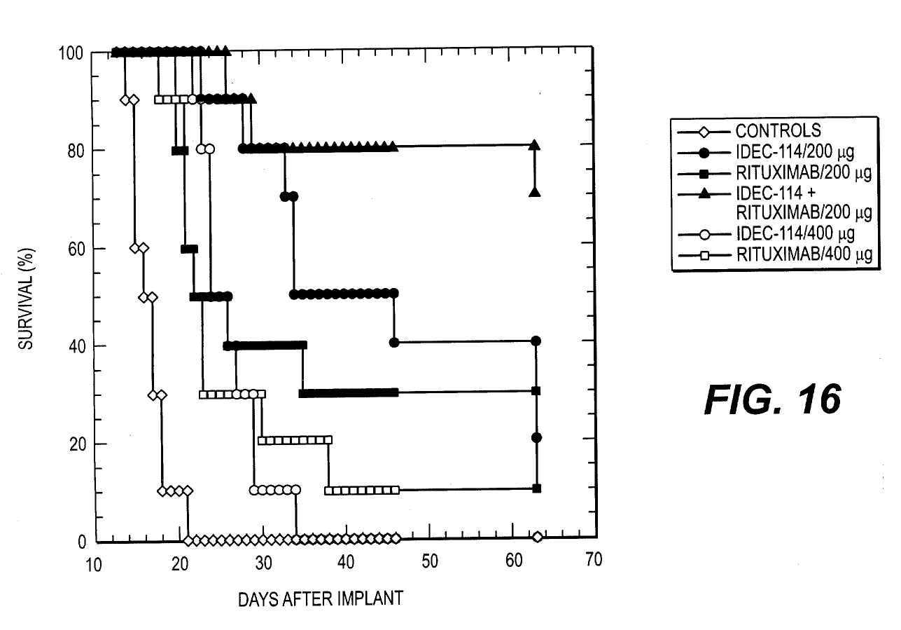

FIG. 16 shows the antitumor response elicited by IDEC-114 and RITUXAN® in SKW/SCID mice. On day 0, groups of mice (N=10) were inoculated intravenously with 4×10⁶ SKW cells. Mice in the combination treatment groups were injected intraperitonealy with 200 μg each of IDEC-114 and rituximab. Mice in the monotherapy treatment groups received 200 ,g or 400 μg of IDEC-114 or rituximab. All antibody injections were given in a final volume of 200 μg. Mice in the control group were injected with a formulation buffer in a volume equal to the antibody injection volume. Antibody injections were given on days 1, 3, 5, 7, 9, and 11 after tumor inoculation. Mice were monitored for disease development and death.

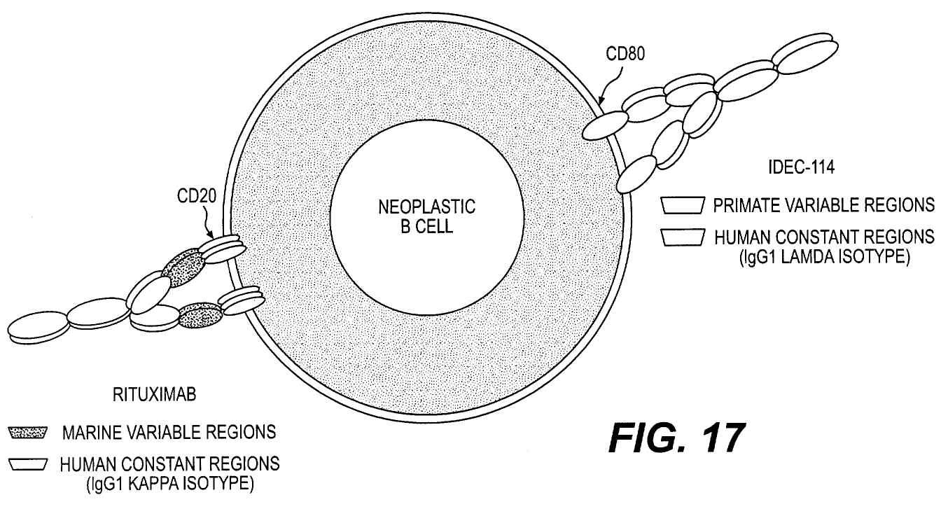

FIG. 17 is a schematic depiction of a neoplastic B cell bound by IDEC-114 and RITUXAN®.

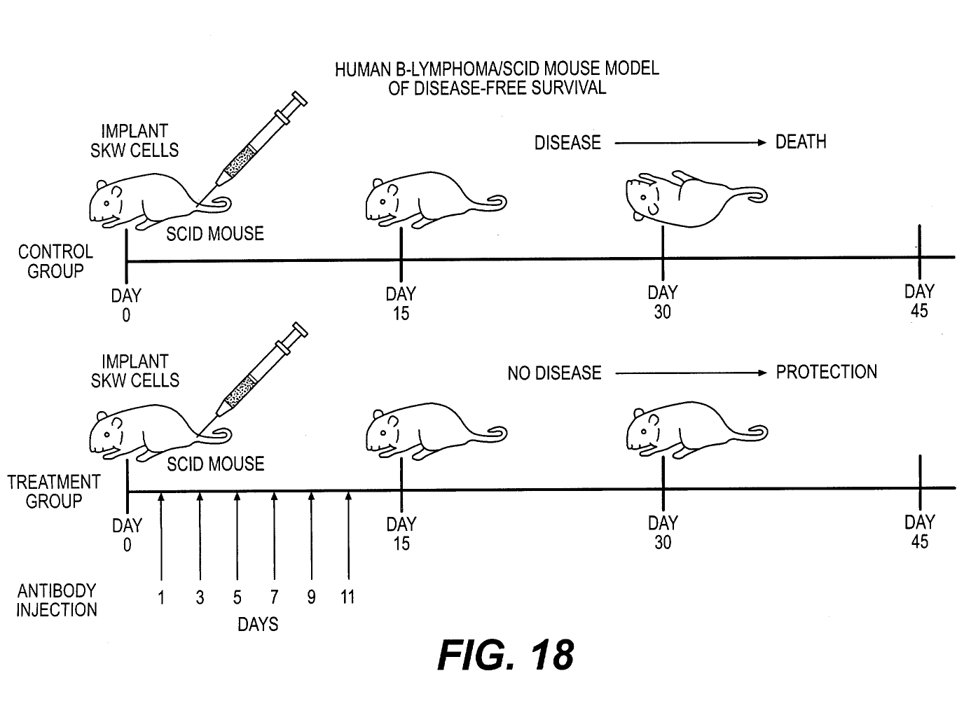

FIG. 18 is schematic depiction of a human B-cell lymphoma/SCID mouse model of disease-free survival and protection after antibody administration.

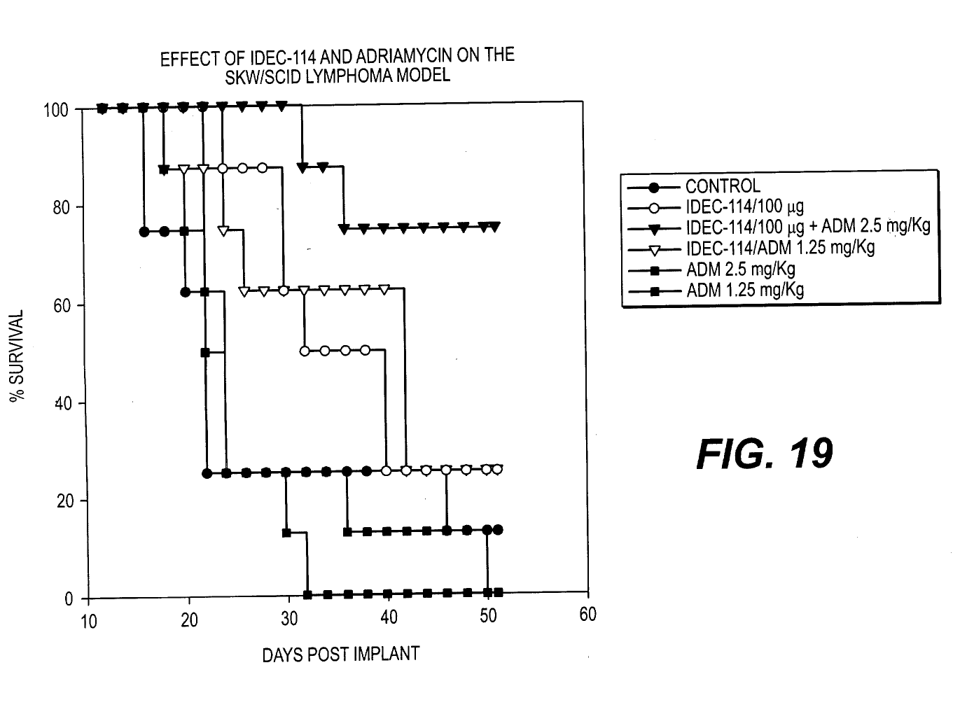

FIG. 19 shows results of combination treatment with IDEC-114 and adriamycin in a human lymphoma/SCID mouse model.

### DETAILED DESCRIPTION OF THE INVENTION

As noted previously, anti-CD80 antibodies have been reported previously as a treatment modality for autoimmune diseases and transplantation. By contrast, as initially described in U.S. Pat. No. 6,113,898, and its divisional applications, which are incorporated by reference in their entirety herein, the present invention is directed toward the use of an anti-CD80 antibody, preferably IDEC-114, for sole or combination therapy for the treatment of B cell lymphoma. In this regard, CD80 is a suitable potential target for lymphoma and other B cell malignancies as it is expressed on malignant B cells. However an anti-CD80 antibody has not previously been demonstrated to be suitable for treatment of B cell lymphoma. Because CD80 is expressed on some malignant B cells, it was theorized that IDEC-114 would be an effective anti-tumor agent against B cell lymphoma cells. Moreover, because CD80 is not expressed in early hematopoietic stem cells, it potentially is an attractive agent for B cell lymphoma therapy as it should not affect B cell and other immune cell proliferation and therefore should not necessitate bone marrow transplant after therapy.

Preclinical safety studies with IDEC-114 in chimpanzees have demonstrated that there are no adverse results with multiple doses up to 30 mg/kg. The only noticeable effect of IDEC-114 in treated chimpanzees was the transient loss of a minor population of CD80+ B cells that is les than 25% of all B cells. In addition, no adverse results have been observed in psoriasis clinical trials. This provides further evidence supportive of the therapeutic use of primatized anti-CD80 antibodies for treatment of B cell lymphoma.

In part, based on these results, experiments were designed to compare the in vitro activity of IDEC-114, rituxmab, and an IDEC-114 rituxmab combinations against B cell lymphoma cells. These experiments (which are disclosed in the examples infra) included the characterization of CD80 expression in lymphoma and leukemia specimens, against B cell lymp, evaluation of the binding activity of IDEC-114 to CD80 expressed on CD80+ CHO cells and lymphoma cell lines, antibody-dependent cellular toxicity (ADCC) activity, complement-dependent cytotoxicity (CDC) activity, and in vivo therapeutic effector treatment in lymphoma.

The results of these experiments, which are discussed in detail in the examples section showed that the tested anti-CD80 antibody, IDEC-114, which is a chimeric anti-CD80 antibody containing primate (cynomolgus macaque) variable regions and human IgG₁ constant regions, and which possesses ADCC and CDC activity, exhibited a synergistic anti-tumor effect against CD80+ cells, particularly B cell lymphoma cells, when used in combination with the tested anti-CD20 antibody, Rituxan®, which is a chimeric anti-CD20 antibody containing rodent variable regions and human IgG₁ constant regions, that has been approved for treatment of non-Hodgkin's lymphoma, and also when used in combination with a chemotherapeutic agent.

While the inventors are unclear as to the exact basis for this synergistic anti-tumor activity, it is believed that the anti-CD20 antibody and/or the chemotherapeutic agent enhances the ADCC response against CD80 positive cells, e.g., B cell lymphoma cells that is elicited by the anti-CD80 antibody. Consequently, the present invention is directed in part to the combined use of an anti-CD80 antibody and an anti-CD20 antibody and/or a chemotherapeutic agent, for treating B cell lymphoma. Also, the invention is directed to the enhancement of the ADCC activity of an anti-CD80 antibody against CD80 positive cells by co-administering this antibody with an anti-CD20 antibody. In the preferred embodiments, the anti-CD80 antibody will comprise IDEC-114 or an anti-CD80 antibody having one or more of the following properties:

(i) it binds the CD80 epitope bound by IDEC-114 or competes with IDEC-114 for binding to CD80 in a binding inhibition assay;

(ii) it does not inhibit the CD80 CTLA-4 interaction;

(iii) it exhibits substantially the same, i.e., at least 75%, more preferably at least 90% the ADCC activity of IDEC-114 against CD80 positive cells e.g., B cell lymphoma cells.

Most preferably, the anti-CD80 antibody will be IDEC-114 or a comparable human, humanized or PRIMATIZED® antibody.

Also, in the preferred embodiments, the anti-CD20 antibody will comprise RITUXAN® or will comprise an anti-CD20 antibody having one or more of the following properties:

(i) it binds the CD20 epitope bound by RITUXAN® or competes with RITUXAN® for binding CD20 in a binding inhibition assay;

(ii) it exhibits substantially the same B cell depleting activity as RITUXAN®, in at least 75% more preferably at least 90% of RITUXAN®, when assayed in vitro or in vivo;

(iii) it exhibits substantially the same apoptive activity against B cells, in particular B cell lymphoma cells as RITUXAN®, i.e. at least 75% that RITUXAN®, and more preferably at least 90% of RITUXAN®; and

(iv) it exhibits substantially the same ADCC and/or CDC activity as RITUXAN®, i.e., at least 75% thereof, and more preferably at least 90% of RITUXAN®.

Most preferably, the anti-CD20 antibody will comprise RITUXAN®, given its established clinical efficacy.

In addition to the anti-CD80 and anti-CD20 antibodies identified above, it may be desirable to use other antibodies in the subject therapies, i.e., other known anti-CD80 or anti-CD20 antibodies, or fragments thereof, and other anti-CD80 or anti-CD20 antibodies or modified antibodies derived from or comprising antigen binding regions of novel antibodies generated using immunization coupled with common immunological techniques. Using art recognized protocols, antibodies can be raised in mammals by multiple subcutaneous or intraperitoneal injections of the relevant antigen (e.g., purified tumor associated antigens or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood. to provide homogenous preparations of monoclonal antibodies (MAbs). Preferably, the lymphocytes are obtained from the spleen.

In this well known process (Kohler et al., Nature, 256:495 (1975)) the relatively short-lived, or mortal, lymphocytes from a mammal which has been injected with antigen are fused with an immortal tumor cell line (e.g. a myeloma cell line), thus producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They therefore produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In other compatible embodiments, DNA encoding the desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be modified as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification thereof by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

Those skilled in the art will also appreciate that DNA encoding other anti-CD80 and anti-CD20 antibodies or antibody fragments may also be derived from antibody phage libraries as set forth, for example, in EP 368 684 B1 and U.S. Pat. No. 5,969,108 each of which is incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. Such procedures provide viable alternatives to traditional hybridoma techniques for the isolation and subsequent cloning of monoclonal antibodies and, as such, are clearly within the purview of the instant invention.

Yet other embodiments of the present invention comprise the generation of substantially human anti-CD20 or anti-CD80 antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array in such germ line mutant mice will result in the production of human antibodies upon antigen challenge. Another preferred means of generating human antibodies using SCID mice is disclosed in commonly-owned, co-pending U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology, 10: 1455-1460 (1992). Specifically, this technique results in the generation of primatized® antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference. As noted already, IDEC-114 is a primatized anti-CD80 antibody, provided according to these references as are the other exemplified anti-CD80 antibodies disclosed in U.S. Pat. No. 6,113,898. It will further be appreciated that the scope of this invention further encompasses all alleles, variants and mutations of the DNA sequences described herein.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligodT cellulose. Techniques suitable to these purposes are familiar in the art and are described in the foregoing references.

cDNAs that encode the light and the heavy chains of the antibody may be made, either simultaneously or separately, using reverse transcriptase and DNA polymerase in accordance with well known methods. It may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells as described herein, restriction mapped and sequenced in accordance with standard, well known techniques set forth in detail in the foregoing references relating to recombinant DNA techniques. Of course, the DNA may be modified according to the present invention at any point during the isolation process or subsequent analysis.

Preferred antibody sequences are disclosed herein in the examples and in the IDEC patents incorporated by reference herein. Oligonucleotide synthesis techniques compatible with this aspect of the invention are well known to the skilled artisan and may be carried out using any of several commercially available automated synthesizers. In addition, DNA sequences encoding several types of heavy and light chains set forth herein can be obtained through the services of commercial DNA synthesis vendors. The genetic material obtained using any of the foregoing methods may then be altered or modified to provide antibodies compatible with the present invention.

A variety of different types of antibodies may be obtained and/or modified according to the instant invention and used in the subject improved therapies for B cell lymphoma and other B cell malignancies. As discussed above, “modified antibodies” according to the present invention are held to mean immunoglobulins, antibodies, or immunoreactive fragments or recombinants thereof, in which at least a fraction of one or more of the constant region domains has been deleted or otherwise altered so as to provide desired biochemical characteristics such as the ability to non-covalently dimerize, increased tumor localization or reduced serum half-life when compared with a whole, unaltered antibody of approximately the same immunogenicity. For the purposes of the instant application, immunoreactive single chain antibody constructs having altered or omitted constant region domains may be considered to be modified antibodies.

Basic immunoglobulin structures in vertebrate systems are relatively well understood. As will be discussed in more detail below, the generic term “immunoglobulin” comprises five distinct classes of antibody that can be distinguished biochemically. While all five classes are clearly within the scope of the present invention, the following discussion will generally be directed to the class of IgG molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

More specifically, both the light and heavy chains are divided into regions of structural and functional homology. The terms “constant” and “variable” are used functionally. In this regard, it will be appreciated that the variable domains of both the light (V_{L}) and heavy (V_{H}) chains determine antigen recognition and specificity. Conversely, the constant domains of the light chain (C_{L}) and the heavy chain (C_{H}1, C^{H}2 or C_{H}3) confer important biological properties such as secretion, transplacental mobility, Fc receptor binding, complement binding, and the like. By convention the numbering of the constant region domains increases as they become more distal from the antigen binding site or amino-terminus of the antibody. Thus, the C_{H}3 and C_{L} domains actually comprise the carboxy-terminus of the heavy and light chains respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages when the immunogobulins are generated either by hybridomas, B cells or genetically engineered host cells. In the heavy chain, the amino acid sequences run from an N-terminus at the forked ends of the Y configuration to the C-terminus at the bottom of each chain. At the N-terminus is a variable region and at the C-terminus is a constant region. Those skilled in the art will appreciate that heavy chains are classified as gamma, mu, alpha, delta, or epsilon, (γ, μ, α, δ, ε) with some subclasses among them. It is the nature of this chain that determines the “class” of the antibody as IgA, IgD, IgE IgG, or IgM. The immunoglobulin subclasses (isotypes) e.g. IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the purview of the instant invention.

In this regard, the variable region may comprise or be derived from any type of mammal that can be induced to mount a humoral response and generate immunoglobulins against CD80 or CD20. As such, the variable region of the modified antibodies may be, for example, of human, murine, non-human primate (e.g. cynomolgus monkeys, macaques, etc.) or lupine origin. In particularly preferred embodiments both the variable and constant regions of compatible modified antibodies are human. In other selected embodiments the variable regions of compatible antibodies (usually derived from a non-human source) may be engineered or specifically tailored to improve the binding properties or reduce the immunogenicity of the molecule. In this respect, variable regions useful in the present invention may be humanized or otherwise altered through the inclusion of imported DNA or amino acid sequences.

For the purposes of the instant application the term “humanized antibody” shall mean an antibody derived from a non-human antibody, typically a murine antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693,762 all of which are hereby incorporated by reference in their entirety.

Those skilled in the art will appreciate that the technique set forth in option (a) above will produce “classic” chimeric antibodies. In the context of the present application the term “chimeric antibodies” will be held to mean any antibody wherein the immunoreactive region or site is obtained or derived from a first species and the constant region (which may be intact, partial or modified in accordance with the instant invention) is obtained from a second species. In preferred embodiments the antigen binding region or site will be from a non-human source (e.g. mouse) and the constant region is human. While the immunogenic specificity of the variable region is not generally affected by its source, a human constant region is less likely to elicit an immune response from a human subject than would the constant region from a non-human source.

Particularly, the variable domains in both the heavy and light chains are altered by at least partial replacement of one or more CDRs and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. It must be emphasized that it may not be necessary to replace all of the CDRs with the complete CDRs from the donor variable region to transfer the antigen binding capacity of one variable domain to another. Rather, it may only be necessary to transfer those residues that are necessary to maintain the activity of the antigen binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that modified antibodies useful in the instant invention will comprise antibodies, or immunoreactive fragments thereof, in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In preferred embodiments, the constant region of the modified antibodies will comprise a human constant region, preferably IgG₁. Modifications to the constant region compatible with the instant invention comprise additions, deletions or substitutions of one or more amino acids in one or more domains. That is, the modified antibodies disclosed herein may comprise alterations or modfications to one or more of the three heavy chain constant domains (C_{H}1, C_{H}2 or C_{H}3) and/or to the light chain constant domain (C_{L}).

As previously indicated, the subunit structures and three dimensional configuration of the constant regions of the various immunoglobulin classes are well known. For example, the C_{H}2 domain of a human IgG Fc region usually extends from about residue 231 to residue 340 using conventional numbering schemes. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 108 residues while the hinge region of an IgG molecule joins the C_{H}2 domain with the C_{H}1 domain. This hinge region encompasses on the order of 25 residues and is flexible, thereby allowing the two N-terminal antigen binding regions to move independently.

Besides their configuration, it is known in the art that the antibody constant region mediates several effector functions. For example, binding of the C1 component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. Although various Fc receptors and receptor sites have been studied to a certain extent, there is still much which is unknown about their location, structure and functioning. As discussed already, it is believed, as shown by the experimental results discussed infra, that the anti-human activity of IDEC-114 is attributable, at least in part to its ADCC activity.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed to produce anti-CD80 and/or anti-CD20 antibodies useful in the subject sole and combination B cell malignancy therapies. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

In particularly preferred embodiments the cloned variable region genes are inserted into an expression vector along with the heavy and light chain constant region genes (preferably human) modified as discussed above. Preferably, this is effected using a proprietary expression vector of IDEC, Inc., referred to as NEOSPLA. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. As seen in the examples below, this vector has been found to result in very high level expression of antibodies upon incorporation of variable and constant region genes, transfection in CHO cells, followed by selection in G418 containing medium and methotrexate amplification. This vector system is substantially disclosed in commonly assigned U.S. Pat. Nos. 5,736,137 and 5,658,570, each of which is incorporated by reference in its entirety herein. This system provides for high expression levels, i.e., >30 pg/cell/day.

In other preferred embodiments the modified antibodies of the instant invention may be expressed using polycistronic constructs such as those disclosed in copending United States provisional application No. 60/331,481 filed Nov. 16, 2001 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of modified antibodies in eukaryotic host cells. Compatible IRES sequences are disclosed in U.S. Pat. No. 6,193,980 which is also incorporated herein. Those skilled in the art will appreciate that such expression systems may be used to effectively produce the full range of modified antibodies disclosed in the instant application.

More generally, once the vector or DNA sequence encoding the antibody or fragment has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

As used herein, the term “transformation” shall be used in a broad sense to refer to any introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. As defined herein, antibodies or modifications thereof produced by a host cell that is, by virtue of this transformation, recombinant. In descriptions of processes for isolation of antibodies from recombinant hosts, the terms “cell” and “cell culture” are used interchangeably to denote the source of antibody unless it is clearly specified otherwise. In other words, recovery of antibody from the “cells” may mean either from spun down whole cells, or from the cell culture containing both the medium and the suspended cells.

The host cell line used for antibody expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In vitro production allows scale-up to give large amounts of the desired antibody. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. As previously described, at least some of the monomeric subunits spontaneously associate non-covalently to form dimeric antibodies. For isolation and recovery of the dimeric antibodies, the immunoglobulins in the culture supernatants may first be concentrated, e.g. by precipitation with ammonium sulphate, dialysis against hygroscopic material such as PEG, filtration through selective membranes, or the like. If necessary and/or desired, the concentrated solutions of tetravalent antibodies are purified by the customary chromatography methods, for example gel filtration, ion-exchange chromatography, chromatography over DEAE-cellulose or (immuno-)affinity chromatography.

Antibody genes can also be expressed non-mammalian cells such as bacteria or yeast. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli; Salmonella; Bacillaceae, such as Bacillus subtilis; Pneumococcus; Streptococcus, and Haemophilus influenzae. It will further be appreciated that, when expressed in bacteria, the immunoglobulin heavy chains and light chains typically become part of inclusion bodies. The chains then must be isolated, purified and then assembled into functional antibodies.

In addition to prokaryates, eukaryotic microbes may also be used. Saccharomyces cerevisiae, or common baker's yeast, is the most commonly used among eukaryotic microorganisms although a number of other strains are commonly available.

For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the trpl gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

Regardless of how clinically useful quantities are obtained, the anti-CD80 or anti-CD20 antibodies used in the therapeutic methods of the present invention may be used in any one of a number of conjugated (i.e. an immunoconjugate) or unconjugated forms. However, unconjugated antibodies are generally preferred given the pronounced ADCC activity of IDEC-114 and RITUXMAN®, which are the preferred embodiments of the invention. In particular, the antibodies of the present invention may be conjugated to cytotoxins such as radioisotopes, therapeutic agents, cytostatic agents, biological toxins or prodrugs. Alternatively, the dimeric antibodies of the instant invention may be used in a nonconjugated or original form to harness the subject's natural defense mechanisms to eliminate the malignant cells. In particularly preferred embodiments, the antibodies may be conjugated to radioisotopes, such as ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re using anyone of a number of well known chelators or direct labeling. In other embodiments, the disclosed compositions may comprise antibodies coupled to drugs, prodrugs or biological response modifiers such as methotrexate, adriamycin, and lymphokines such as interferon. Still other embodiments of the present invention comprise the use of antibodies conjugated to specific biotoxins such as ricin or diptheria toxin. In yet other embodiments the modified antibodies may be complexed with other immunologically active ligands (e.g. antibodies or fragments thereof) wherein the resulting molecule binds to both the neoplastic cell and an effector cell such as a T cell. The selection of which conjugated or unconjugated modified antibody to use will depend of the type and stage of cancer, use of adjunct treatment (e.g., chemotherapy or external radiation) and patient condition. It will be appreciated that one skilled in the art could readily make such a selection in view of the teachings herein.

As used herein, “a cytotoxin or cytotoxic agent” means any agent that is detrimental to the growth and proliferation of cells and may act to reduce, inhibit or distroy a cell or malignancy when exposed thereto. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. As will be discussed in more detail below, radionuclide cytotoxins are particularly preferred for use in the instant invention. However, any cytotoxin that acts to retard or slow the growth of immunoreactive cells or malignant cells or to eliminate these cells and may be associated with the antibodies disclosed herein is within the purview of the present invention.

It will be appreciated that, in previous studies, anti-tumor antibodies labeled with these isotopes have been used successfully to destroy cells in solid tumors as well as lymphomas/leukemias in animal models, and in some cases in humans. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

It will be appreciated that, in previous studies, anti-tumor antibodies labeled with isotopes have been used successfully to destroy cells in solid tumors as well as lymphomas/leukemias in animal models, and in some cases in humans. The radionuclides act by producing ionizing radiation which causes multiple strand breaks in nuclear DNA, leading to cell death. The isotopes used to produce therapeutic conjugates typically produce high energy α-, γ- or β-particles which have a therapeutically effective path length. Such radionuclides kill cells to which they are in close proximity, for example neoplastic cells to which the conjugate has attached or has entered. They generally have little or no effect on non-localized cells. Radionuclides are essentially non-immunogenic.

With respect to the use of radiolabeled conjugates in conjunction with the present invention, the antibodies may be directly labeled (such as through iodination) or may be labeled indirectly through the use of a chelating agent. As used herein, the phrases “indirect labeling” and “indirect labeling approach” both mean that a chelating agent is covalently attached to an antibody and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyidiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a dimeric antibody (typically via an amino acid residue). More specifically, these linking technologies include random labeling and site-directed labeling. In the latter case, the labeling is directed at specific sites on the antibody, such as the N-linked sugar residues present only on the Fc portion of the conjugates. Further, various direct labeling techniques and protocols are compatible with the instant invention. For example, Technetium-99m labelled antibodies may be prepared by ligand exchange processes, by reducing pertechnate (TcO₄⁻) with stannous ion solution, chelating the reduced technetium onto a Sephadex column and applying the antibodies to this column, or by batch labelling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is 131I covalently attached via tyrosine residues. Modified antibodies according to the invention may be derived, for example, with radioactive sodium or potassium iodide and a chemical oxidising agent, such as sodium hypochlorite, chloramine T or the like, or an enzymatic oxidising agent, such as lactoperoxidase, glucose oxidase and glucose. However, for the purposes of the present invention, the indirect labeling approach is particularly preferred.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein in their entirety. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation in co-pending application Ser. Nos. 08/475,813, 08/475,815 and 08/478,967, incorporated by reference in their entirety herein, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, antibodies may be conjugated to different radiolabels for diagnostic and therapeutic purposes. To this end the aforementioned co-pending applications, herein incorporated by reference in their entirety, disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the art are credited with the ability to readily determine which radionuclide is most appropriate under various circumstances. For example, ¹³¹I is a well known radionuclide used for targeted immunotherapy. However, the clinical usefulness of 131I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for ttaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target antigen.

Effective single treatment dosages (i.e., therapeutically effective amounts) of ⁹⁰Y-labeled modified antibodies range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, ⁴⁷Rh, ¹⁰⁵Rh, ¹⁰⁹Pd, ¹⁵³Sm, ¹⁸⁸Re, ¹⁹⁹Au, ²²⁵Ac ²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy Peirersz et al. Immunol. Cell Biol. 65: 111-125 (1987). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

In addition to radionuclides, the antibodies of the present invention may be conjugated to, or associated with, any one of a number of biological response modifiers, pharmaceutical agents, toxins or immunologically active ligands. Those skilled in the art will appreciate that these non-radioactive conjugates may be assembled using a variety of techniques depending on the selected cytotoxin. For example, conjugates with biotin are prepared e.g. by reacting the antibodies with an activated ester of biotin such as the biotin N-hydroxysuccinimide ester. Similarly, conjugates with a fluorescent marker may be prepared in the presence of a coupling agent, e.g. those listed above, or by reaction with an isothiocyanate, preferably fluorescein-isothiocyanate. Conjugates of the antibodies of the invention with cytostatic/cytotoxic substances and metal chelates are prepared in an analogous manner.

Preferred agents for use in the present invention are cytotoxic drugs, particularly those which are used for cancer therapy. Such drugs include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. As noted previously, one skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that antibodies can also be associated with a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the antibodies of the present invention comprise cytokines such as lymphokines and interferons. In view of the instant disclosure it is submitted that one skilled in the art could readily form such constructs using conventional techniques.

Another class of compatible cytotoxins that may be used in conjunction with the disclosed antibodies are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked modified antibodies would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

As discussed above, preferred embodiments of the invention comprise the administration of an anti-CD80 antibody preferably one having ADCC activity, to a patient with a B cell malignancy, e.g., leukemia or lymphoma or in combination or conjunction with one or more other therapies such, in particular anti-CD20 antibody therapy, and/or chemotherapy or radiotherapy (i.e. a combined therapeutic regimen). As used herein, the administration of antibodies in conjunction or combination with an adjunct therapy means the sequential, simultaneous, coextensive, concurrent, concomitant or contemporaneous administration or application of the therapy and the subject anti-CD80 and/or anti-CD20 antibodies. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated antibodies could be administered intravenously followed by tumor localized external beam radiation. In yet other embodiments, the antibody may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of the anti-CD80 antibody (with or without cytotoxin) and a chemotherapeutic agent may be administered in any order and within any time frame that provides a therapeutic benefit to the patient. That is, the chemotherapeutic agent and anti-CD80 antibody may be administered in any order or concurrently. In selected embodiments the antibodies of the present invention will be administered to patients that have previously undergone chemotherapy. In yet other embodiments, the antibodies and the chemotherapeutic treatment will be administered substantially simultaneously or concurrently. For example, a B cell lymphoma patient may be given the anti-CD80 antibody while undergoing a course of chemotherapy. In preferred embodiments the modified antibody will be administered within 1 year of any chemotherapeutic agent or treatment. In other preferred embodiments the ant-CD80 antibody will be administered within 10, 8, 6, 4, or 2 months of any chemotherapeutic agent or treatment. In still other preferred embodiments the dimeric antibody will be administered within 4, 3, 2 or 1 week of any chemotherapeutic agent or treatment. In yet other embodiments the dimeric antibody will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

It will further be appreciated that the anti-CD80 antibodies used in the instant invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow B reserves. In other preferred embodiments the radiolabeled immunoconjugates disclosed herein may be effectively used with radiosensitizers that increase the susceptibility of the neoplastic cells to radionuclides. For example, radiosensitizing compounds may be administered after the radiolabeled modified antibody has been largely cleared from the bloodstream but still remains at therapeutically effective levels at the site of the tumor or tumors.

With respect to these aspects of the invention, exemplary chemotherapic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChIVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in Harrison's Principles of Internal Medice 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more anti-CD80 or anti-CD20 antibodies as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade NHL, who fail to achieve remission or relapse, salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the antibodies of the instant invention may vary by subject or may be administered according to what is known in the art. See for example, Bruce A Chabner et al., Antineoplastic Agents, in Googman & Gilman's the Pharmacological Basis of Therapeutics 1233-1287 ((Joel G. Hardman et al., eds., 9^{th} ed. 1996).

As previously discussed, the antibodies of the present invention, immunoreactive fragments or recombinants thereof are administered in a pharmaceutically effective amount for the in vivo treatment of B cell malignancies, particularly leukemias and lymphomas. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of the dimeric antibody, immunoreactive fragment or recombinant thereof, conjugated or unconjugated to a therapeutic agent, shall be held to mean an amount sufficient to achieve effective binding with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the antibody.

More specifically, the subject therapies should be useful for reducing tumor size, inhibiting tumor growth and/or prolonging the survival time of tumor-bearing animals. Accordingly, this invention also relates to a method of treating tumors in a human or other animal by administering to such human or animal an effective, non-toxic amount of antibody. One skilled in the art would be able, by routine experimentation, to determine what an effective, non-toxic amount of antibody would be for the purpose of treating CD80 positive malignancies. For example, a therapeutically active amount of a antibody may vary according to factors such as the disease stage (e.g., stage I versus stage IV), age, sex, medical complications (e.g., immunosuppressed conditions or diseases) and weight of the subject, and the ability of the antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

For purposes of clarification “Mammal” refers to any animal classified as a mammal, including humans, domestic and farm animals, and zoo, sports, or pet animals, such as dogs, horses, cats, cows, etc. Preferably, the mammal is human.

“Treatment” refers to both therapeutic treatment and prophylactic or preventative measures. Those in need of treatment of a B cell malignancy e.g., B cell lymphoma, include those already with the disease or disorder as well as those in which the disease or disorder is to be prevented. Hence, the mammal may have been diagnosed as having the disease or disorder or may be predisposed or susceptible to the disease.

In keeping with the scope of the present disclosure, the CD80 and CD20 antibodies of the invention may be administered to a human or other animal in accordance with the aforementioned methods of treatment in an amount sufficient to produce such effect to a therapeutic or prophylactic degree. The antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of dimeric antibodies according to the present invention may prove to be particularly effective.

Methods of preparing and administering conjugates of the antibody, immunoreactive fragments or recombinants thereof, and a therapeutic agent are well known to or readily determined by those skilled in the art. The route of administration of the antibody or antibodies (or fragment thereof) of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a preferred administration form would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumine), etc. However, in other methods compatible with the teachings herein, the dimeric antibodies can be delivered directly to the site of the adverse cellular population thereby increasing the exposure of the diseased CD80 positive tissue to the therapeutic agent.

Preparations for parenteral administration includes sterile aqueous or non-tumor aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a dimeric antibody by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to B cell neoplastic disorders.

As discussed in detail above, preferred embodiments of the present invention provide compounds, compositions, kits and methods for the treatment of neoplastic B cell disorders in a mammalian subject in need of treatment thereof. Preferably, the subject is a human. The B cell neoplastic disorder (e.g., cancers and malignancies) may comprise solid tumors and preferably will comprise hematologic malignancies such as lymphomas and leukemias. In general, the disclosed invention may be used to prophylactically or therapeutically treat any neoplasm comprising CD80 that allows for the targeting of the antibody to the cancerous cells.

Exemplary hematologic malignancies that are amenable to treatment according to the disclosed invention include Hodgkins and non-Hodgkins lymphoma as well as leukemias, including ALL-L3 (Burkitt's type leukemia), chronic lymphocytic leukemia (CLL) and monocytic cell leukemias. It will be appreciated that the antibodies and compounds and methods of the present invention are particularly effective in treating a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention. In addition to the aforementioned neoplastic disorders, it will be appreciated that the disclosed invention may advantageously be used to treat additional malignancies bearing compatible tumor associated antigens.

### EXAMPLES

### Example 1

Recombinant immunoglobulin libraries displayed on the surface of filamentous phage were first described by McCafferty et al, Nature, 348:552-554, 1990 and Barbas et al, Proc. Natl. Acad. Sci., USA 88:7978-7982, 1991. Using this technology, high affinity antibodies have been isolated from immune human recombinant libraries (Barbas et al, Proc. Natl. Acad. Sci., USA 589:10164-10168, 1992). Although the phage display concept used is substantially similar to that described by Barbas, 1991, Id. the technique has been modified by the substitution of a unique vector for monkey libraries to reduce the possibility of recombination and improve stability. This vector, pMS, FIG. 1 contains a single lac promoter/operator for efficient transcription and translation of polycistronic heavy and light chain monkey DNA. This vector contains two different leader sequences, the omp A (Movva et al, J. Biol. Chem., 255: 27-29, (1980), for the light chain and the pel B (Lei, J. Bact., 4379-109:4383 (1987) for the heavy chain Fd. Both leader sequences are translated into hydrophobic signal peptides that direct the secretion of the heavy and light chain cloned products into the periplasmic space. In the oxidative environment of the periplasm, the two chains fold and disulfide bonds form to create stable Fab fragments. We derived the backbone of the vector from the phagemid bluescript. (Stratagene, La Jolla, Calif.). It contains the gene for the enzyme beta-lactamase that confers ampicillin (carbenicillin) resistance to bacteria that harbor pMS DNA. We also derived, from bluescript, the origin of replication of the multicopy plasmid ColEl and the origin of replication of the filamentous bacteriophage f1. The origin of replication of phage f1 (the so-called intragenic region), signals the initiation of synthesis of single stranded pMS DNA, the initiation of capsid formation and the termination of RNA synthesis by viral enzymes. The replication and assembly of pMS DNA strands into phage particles requires viral proteins that must be provided by a helper phage. We have used helper phage VCSM13 which is particularly suited for this, since it also contains a gene coding for kanamycin resistance. Bacteria infected with VCSM13 and pMS can be selected by adding both kanamycin and carbenicillin to the growth medium. The bacteria will ultimately produce filamentous phage particles containing either pMS or VCSM13 genomes. Packaging of the helper phage is less efficient than that of pMS, resulting in a mixed phage population that contains predominately recombinant pMS phages. The ends of the phage pick up minor coat proteins specific to each end. Of particular interest here is the gene III product which is present in three to five copies at one end of the phage. The gene III product is 406 amino acid residues and is required for phage infection of E. coli via the F pili. The first two domains of the heavy chain, the variable and the CH1 domain, are fused to the carboxy-terminal half of the gene III protein. This recombinant pili protein, directed by the pel B leader, is secreted to the peroplasm where it accumulates and forms disulfide bonds with the light chain before it is incorporated in the coat of the phage. Also, another vector contains a FLAG sequence engineered downstream of the gene III . The FLAG is an 8 amino acid peptide expressed at the carboxy terminal of the Fd protein. We are using commercially available monoclonal anti-FLAG M2 for both purification and detection of phage Fab by ELISA (Brizzard, Bio Techniques, 16(4):730-731, (1994)).

After constructing the vector pMS, we tested its ability to produce phage bound Fab using control antibody genes. We cloned an anti-tetanus toxoid antibody, (obtained from Dr. Carlos Barbas), into pMS and transformed XLI-blue. We co-infected our cells with VCSM13 and generated phage displaying the anti-tetanus toxoid antibody. We performed efficiency experiments where anti-tetanus toxoid phage were combined with phage beading an irrelevant antibody at 1:100,000. We performed three rounds of panning by applying 50 μl of the mixed phage to antigen (tetanus toxoid) coated polystyrene wells. Non-adherent phage were washed off and the adherent phage were eluted with acid. The eluted phage were used to infect a fresh aliquot of XL1 Blue bacteria and helper phage was added. After overnight amplification, phage were prepared and again panned on antigen coated plates. After three rounds of panning, we were able to show that we had successfully enriched for the anti-tetanus toxoid phage. The success of this technology also depends on the ability to prepare soluble Fabs for characterization of the final panned product. This was achieved by excising gene III from the pMS DNA using the restriction enzyme Nhe I followed by re-ligation. After the gene III was excised, the Fab was no longer displayed on the phage surface but accumulated in the piroplasmic space. Lysates were prepared from bacteria expressing soluble Fab and tested for antigen specificity using an ELISA. High levels of soluble Fab were detected.

In order to adapt phage display technology for use with macaque libraries, we developed specific primers for PCR amplifying monkey immunoglobulin genes. These were based on macaque sequences we obtained while developing the PRIMATIZED™ antibody technology (See, 08/379,072, incorporated by reference herein) and databases containing human sequences. (Kabat et al, (1991), “Sequences of Proteins of Immunological Interest,” U.S. Dept. of Health and Human Services, National Institute of Health).

We developed three sets of primers to cover amplification of the macaque repertoire. Our first set of primers was designed for amplification of the heavy chain VH and CH1 (Fd) domains. It consisted of a 3′ CH1 domain primer and six 5′ VH family specific primers that bind in the framework 1 region. Our second set of primers, for amplifying the whole lambda chain, covers the many lambda chain subgroups. It consists of a 3′ primer and three 5′ degenerate primers that bind in the VL framework 1 region. Our third set of primers was designed for amplification of the kappa chain subgroups. It consists of one 3′ primer and five VK framework 1 primers. Using each of these sets, PCR parameters were optimized to obtain strong enough signals from each primer pair so that ample material was available for cloning of the library. We recently created macaque combinatorial libraries in our pMS vector using these optimized PCR conditions. Bone marrow biopsies were taken from CD4 immune monkeys as the source of immunoglobulin RNA. The libraries contained approximately 10⁶ members and are currently being panned for specific binders on antigen coated wells.

### Example 2

Development of B7/CTLA-4 Reagents

We have generated a number of reagents for the purpose of immunizing monkeys, developing binding and functional assays in vitro, screening heterohybridomas and panning phage libraries. Table 1 lists each reagent and its intended purpose. In the case of B7.1, RNA was extracted from SB cells and converted to cDNA using reverse transcriptase. The first strand cDNA was PCR amplified using B7.1 specific primers and cloned into IDEC's NEOSPLA mammalian expression vectors. CHO cells were transfected with B7.1 NEOSPLA DNA and clones expressing membrane associated B7.1 were identified. The B7.1 fusion protein was generated similarly, except that the PCR amplified B7.1 gene was cloned into a NEOSPLA cassette vector containing the human CH2 and CH3 immunoglobulin genes. CHO cells were transformed with the B7.1/Ig NEOSPLA DNA and stable clones secreting B7.1/Ig fusion protein were amplified. In general, the B7.2 and CTLA4 reagents were generated in the same manner, except that for B7.2 the RNA was isolated from human spleen cells that had been stimulated 24 hours with anti-Ig and IL-4, and for the CTLA4 constructs the gene source was PHA activated human T cells.

TABLE 1

| Reagent | Purpose | CHO Expression |
|---|---|---|
| Soluble B7.1 | Immunization, immunoassays | Yes |
| B7.1 Transfectant | Screening, ELISA | Yes |
| B7.1/Ig Fusion Protein | Inhibition studies, panning | Yes |
| B7.2 Transfectant | Screening, ELISA | Yes |
| B7.2/Ig Fusion Protein | Inhibition studies, panning | To be completed |
| CTLA4 Transfectant | Inhibition studies | To be completed |
| CTLA4/Ig | Inhibition studies | To be completed |

The availability of these reagents, together with monoclonal antibodies to B7.1 (L3074) (Becton Dickinson, 1994) and B7.2 (Fun-1 (Engel et al, Blood, 84, 1402-1407, (1994) and purified goat and rabbit antisera, specifically developed to detect monkey Fab fragments, facilitates identification of antibodies having the desired properties.

### Example 3

Investigation of the Immune Response in Cynomolgus Monkeys to Soluble and Cell Associated Human B7.1

To evaluate the feasibility of producing monkey antibodies to human B7.1 antigen, we first purified recombinant SB7.1 from CHO cell media by affinity chromatography using a L307.4-sepharose affinity column. SB7.1 was then injected, with adjuvant, into five mature cynomolgus macaques. After a 3 to 4 month period of booster immunizations, sera from the monkeys immunized with SB7.1 or human SB cells were tested for antigen binding.

Serum samples from the five monkeys immunized with SB7.1. and three additional animals immunized with B7.1 positive human SB cells, were tested for antibody titers against membrane associated B7.1 expressed in transfected CHO cells. The results summarized in FIG. 3 showed that four out of five monkeys immunized with affinity-purified SB7.1 produced antibody titers in excess of 1:5000. The three animals immunized with SB cells containing cell associated B7.1 expressed lower titers of antibodies ranging from 1:1400 to 1:2800.

### Example 4

We purified antibodies from sera of all eight immunized monkeys using SB7.1-sepharose and then tested their ability to bind to 1) SB7.1 coated plates in ELISA; 2) antigen positive B cells and 3) B7.1 CHO transfectomas. In addition, they were evaluated for their ability to block B cell interactions as measured by IL-2 production and tritiated thymidine uptake in a mixed lymphocyte reaction (MLR). For T cell binding experiments, human buffy coat peripheral blood lymphocytes were cultured for 3-6 days in the presence of PHA stimulator. B7 binding was detected by radio assay using ¹²⁵I-radiolabeled soluble B7.1 (SB7.1).

### Example 5

Direct Binding of Monkey Antibodies to Radiolabeled SB7.1

¹²⁵I radiolabeled SB7.1 was tested for binding to anti-B7.1 antibodies at 4, 1 and 0.25 μg/ml in solution. The results shown in Table 2 suggest that most of the antibodies produced by monkeys immunized with SB7.1 were capable of binding the affinity-purified ¹²⁵I-SB7.1 in a concentration dependent manner. To evaluate the specificity of binding to labeled SB7.1, unlabelled SB7.1 competition experiments were done with antibodies from two animals. Affinity-purified antibodies from monkeys 1133 and 1144 were coated onto microwell plates at 400 ng/well. Affinity-purified unlabeled SB7.1 (500 and 100 ng/well) was used as competitor. The results shown in FIG. 4 demonstrated that SB7.1 preparations are effective in inhibiting the ¹²⁵I-SB7.1 from binding to the antibodies.

TABLE 2
Binding of SB7-I¹²⁵ to Monkey Antibodies Affinity
Purified on a SB7-Sepharose Affinity Column

| Antibody | Monkey Numbers | | | | | | | |
|---|---|---|---|---|---|---|---|---|
| (μg/ml) | 769 | 908 | 1133 | 1135 | 1137 | 1139 | 1144 | 1146 |
| 4 | 175 | 213 | 9,056 | 12,771 | 4,318 | 226 | 5,781 | 108 |
| 1 | 106 | 142 | 6,569 | 7,940 | 3,401 | 110 | 3,901 | 80 |
| 0.25 | 95 | 104 | 1,803 | 2,673 | 1,219 | 100 | 1,186 | 94 |

Data are mean values of duplicate assays and represent cpm SB7-1¹²⁵ bound.

### Example 6

Direct Binding of Radiolabeled Affinity-purified Monkey Antibodies to B7⁺ Cells and Inhibition by SB7.1.

Affinity-purified radiolabeled monkey anti-B7.1 antibodies from monkey PRI135 were compared with radiolabeled L307.4 MAb for direct binding to B7 positive human SB cells. As a specificity control, unlabeled SB7.1 (0.002-20 μg/mi) was added to compete with both radiolabeled antibodies. We demonstrated that monkey antibodies can bind cell associated B7.1 and are inhibited with SB7.1, as shown in FIG. 5. Inhibition as high as 90% was observed with SB7.1.

### Example 7

Direct Binding of Radiolabeled B7-Ig Fusion Protein to Activated T Cells and Inhibition by Affinity-purified Monkey Antibodies

Human peripheral blood T lymphocytes were activated for 3-6 days and tested for direct binding of ¹²⁵I-B7.1-Ig. Because of Fc receptor upregulation on activated human T cells, it was necessary to pre-incubate the cells with heat-aggregated pre-immune immunoglobulin to block Fc binding sites prior to addition of B7.1-Ig to the cells. A background control using SP2/0 murine myeloma cells was included to allow correction of the background binding. FIG. 6 shows that inhibition of ¹²⁵I-B7.1-Ig fusion protein binding to activated T cells was achieved with affinity-purified monkey antibodies at concentrations from 200 to 8 μg/mi. Unlabeled SB7.1 and L307.4 MAb used as controls were also effective in inhibiting B7.1-Ig fusion protein cell binding.

### Example 8

Inhibition of IL-2 Production in Mixed Lymphocyte Reactions by Monkey Anti-B7 Antibodies

The blocking of CD28/B7 interaction leads to inhibition of IL-2 production by T lymphocytes. In the experiment shown in FIG. 7, affinity-purified monkey antibodies from two monkeys immunized with SB7.1 (monkeys 1137 and 1135) and one immunized with B7 positive SB cells (monkey 1146) were evaluated for their abilities to inhibit human T cell activation in mixed lymphocyte reaction (MLR), as measured by inhibition of IL-2 production. The results of this experiment show that affinity-purified anti-B7.1 antibodies from monkeys 1146 and 1137 inhibited IL-2 production when added at concentrations of 50 μg/ml. Monkey 1135 antibodies could not be evaluated at the two highest concentrations due to lack of material, yet gave significant inhibition at lower concentrations. The murine MAb L307.4 was inhibitory at concentrations of 10 μg/ml. Other monkey sera tested at these concentrations were negative (data not shown). These results demonstrate that at least three of the monkeys immunized with both soluble and membrane associated forms of the B7 antigen are producing B7-blocking antibodies with immunosuppressive potential.

### Example 9

Investigation of Cross-reactivity in B7.1 Immunized Monkey Serum to B7.2 Antigen.

Antibodies raised against B7.1 are to be tested for cross-reactivity to B7.2Ig. Preliminary results using B7.1 affinity-purified antibodies from B7.1 immune sera provided suggestive evidence of binding to B7.2 transfected CHO cells (not shown). These data should be confirmed by using soluble B7.2Ig reagents. We will first purify additional monkey antibodies from B7.1 immunized animals by affinity chromatography on B7.1Ig-sepharose. We will then produce and purify B7.2Ig from CHO cells in sufficient quantities to prepare a B7.2Ig-sepharose affinity column. We will select from the B7.1 specific antibody population those antibodies which cross-react with B7.2 by binding to the B7.2Ig-sepharose column. Any cross-reactive antibodies identified will be further characterized by direct binding to both B7.1 and B7.2 transfected CHO cells and inhibition of binding to B7.2 transfected cells by B7.1Ig.

### Example 10

Generation of a Phage Display Library

Recombinant phage display libraries are generated from B7.1 and B7.2 immune monkeys. Lymph node and bone marrow biopsies are performed 7-12 days after immunization to harvest RNA rich B cells and plasma cells. RNA is isolated from the lymphocytes using the method described by Chomczynski Anal. Biochem., 162(1), 156-159, (1987). RNA is converted to cDNA using an oligo dT primer and reverse transcriptase. The first strand cDNA is divided into aliquots and PCR amplified using the sets of kappa, lambda, and heavy chain Fd region primers described earlier and either Pfu polymerase (Stratagene, San Diego) or Taq polymerase (Promega, Madison). The heavy chain PCR amplified products are pooled, cut with Xho VSpe I restriction enzymes and cloned into the vector pMS. Subsequently, the light chain PCR products are pooled, cut with Sac I/Xba I restriction enzymes, and cloned to create the recombinant library. XLI-Blue E. coli is transformed with the library DNA and super-infected with VCSM13 to produce the phage displaying antibodies. The library is panned four rounds on polystyrene wells coated with B7.1 or B7.2 antigen. Individual phage clones from each round of panning are analyzed. The pMS vector DNA is isolated and the gene III excised. Soluble Fab fragments are generated and tested in ELISA for binding to B7.1 and B7.2.

### Example 11

Characterization of Phage Fab Fragments

The monkey phage Fab fragments are characterized for their specificity and the ability to block B7.1-Ig and B7.2-Ig binding to CTLA-4-Ig or CTLA-4 transfected cells. Phage fragments are also characterized for cross-reactivity after first panning for 4 rounds on the B7 species used for immunization in order to select for high affinity fragments. Fab fragments identified from four rounds of panning either on B7.1 or B7.2 antigen coated surfaces are scaled up by infection and grown in 24 hour fermentation cultures of E coli. Fragments are purified by Kodak FLAG binding to a anti-FLAG affinity column. Purified phage Fabs are tested for affinity by an ELISA based direct binding modified Scatchard analysis (Katoh et al, J. Chem. BioEng., 76:451-454, (1993)) using Goat anti-monkey Fab antibodies or anti-FLAG MAb conjugated with horseradish peroxidase. The anti-monkey Fab reagents will be absorbed against human heavy chain constant region Ig to remove any cross-reactivity to B7-Ig. Kd values are calculated for each fragment after measurements of direct binding to B7.1-Ig or B7.2-Ig coated plates.

### Example 12

Phage Fab Fragment Blocking of CTLA-4/B7 Binding

Fab fragments most effectively blocking the binding of B7-Ig at the lowest concentrations are selected as lead candidates. Selections are made by competing off ¹²⁵I-B7-Ig binding to CTLA-4-Ig or CTLA-4 transfected cells. Additional selection criteria include, blocking of mixed lymphocyte reaction (MLR), as measured by inhibiting 3H-thymidine uptake in responder cells (Azuma et al, J. Exp. Med., 177:845-850,; Azuma et al, Nature, 301:76-79, (1993)) and direct analysis of IL-2 production using IL-2 assay kits. The three or four candidates which are most effective in inhibiting of MLR and CTLA-4 binding assays are chosen for cloning into the above-described mammalian expression vector for transfection into CHO cells and expression of chimeric monkey/human antibodies.

### Example 13

Generation of Monkey Heterohybridomas

Monkey heterohybridomas secreting monoclonal antibodies are generated from existing immunized animals whose sera tested positive for B7.1 and/or B7.2. Lymph node biopsies are taken from animals positive to either, or both, antigens. The method of hybridoma production is similar to the established method used for the generation of monkey anti-CD4 antibodies (Newman, 1992(Id.)). Monkeys with high serum titers will have sections of inguinal lymph nodes removed under anesthesia. Lymphocytes are washed from the tissue and fused with KH6/B5 heteromyeloma cells (Carrol et al, J. Immunol. Meth., 89:61-72, (1986)) using polyethylene glycol (PEG). Hybridomas are selected on H.A.T. media and stabilized by repeated subcloning in 96 well plates.

Monkey monoclonal antibodies specific for B7.1 antigen are screened for cross-reactivity to B7.2. Monkey anti-B7 antibodies will be characterized for blocking of B7/CTLA-4 binding using the ¹²⁵I-B7-Ig binding assay. Inhibition of MLR by 3H-Thymidine uptake and direct measurement of IL-2 production is used to select three candidates. Two candidates will be brought forward in Phase II studies and expressed in CHO cells while repeating all functional studies. For the purposes of developing an animal model for in vivo pharmacology, anti-B7 antibodies will be tested on cells of several animal species. The establishment of an animal model will allow preclinical studies to be carried out for the selected clinical indication.

### Example 14

As discussed supra, using the above heterohybridoma methods, 4 lead monkey anti-B7.1 antibodies have been identified: 16C10, 7B6, 7C10 and 20C9. These antibodies were characterized as follows:

To demonstrate the monkey antibodies' ability to block the physical interaction between CTLA4-Ig, varying concentrations of the monkey anti-B7.1 antibodies and unlabeled CTLA4-Ig were incubated with ¹²⁵1-radiolabeled CTLA4-Ig. The results of the inhibition assay showed that the IC50 (the concentration of inhibitor which results in 50% inhibition) for the monkey antibodies are:

| | | |
|---|---|---|
| a: | 7C10: | 0.39 μg/MI |
| b: | 16C10: | 1.60 μg/MI |
| c: | 20C9: | 3.90 μg/MI |
| d: | 7B6: | 39.0 μg/MI |

Scatchard analysis showed that the apparent affinity constants (Kd) for the monkey antibodies binding to B7-Ig coated plates were approximated to be:

| | | |
|---|---|---|
| a: | 7C10: | 6.2 × 10⁻⁹M |
| b: | 16C10: | 8.1 × 10⁻⁹M |
| c: | 7B6: | 10.7 × 10⁻⁹M |
| d: | 20C9: | 16.8 × 10⁻⁹M |

The antibodies were tested in vitro in a mixed lymphocyte reaction assay (MLR). The MLR showed that all 4 anti-B7.1 antibodies inhibit IL-2 production to different extents:

| | | |
|---|---|---|
| a: | 7B6: | 5.0 μg/MI |
| b: | 16C10: | 0.1 μg/MI |
| c: | 20C9: | 2.0 μg/MI |
| d: | 7C10: | 5.0 μg/MI |

The monkey anti-B7.1 antibodies were tested for their ability to bind B7 on human peripheral blood lymphocytes (PBL). FACS analysis showed that all 4 monkey antibodies tested positive.

Monkey antibodies 16C10, 7B6, 7C10 and 20C9 were tested for C1q binding by FACS analysis. Results showed 7C10 monkey Ig had strong human C1q binding after incubating with B7.1 CHO-transfected cells. 16C10 was negative, as were the 20C9 and 7B6 monkey antibodies.

### Example 15

Using the primatized antibody methodology incorporated by reference to commonly assigned U.S. Ser. No. 08/379,072, and using the NEOSPLA vector system shown in FIG. 2, the heavy and light variable domains of 7C10, 7B6 and 16C10 were cloned and primatized forms thereof have been synthesized in CHO cells using the NEOSPLA vector system. The amino acid and nucleic acid sequences for the primatized 7C10 light and heavy chain, 7B6 light and heavy chain, and 16C10 light and heavy chain are respectively shown in FIGS. 8a, 8b, 9a, 9b, 10a and 10b.

The following materials and methods were used in the examples which follow.

Materials and Methods

Cell Lines

CD20- and B7-expressing B-lymphoma cell lines (SKW, SB, and Daudi cells) were cultured in complete medium. Complete medium is RPMI 1640 medium (Irvine Scientific, Santa Ana, Calif.) supplemented with 10% heat inactivated FBS (Hyclone), 2 mM I-glutamine, 100 units/ml of penicillin, and 100 ug/ml of streptomycin. The SKW cell line is Epstein-Barr virus (EBV) positive and can be induced to secrete IgM (SKW 6.4, ATCC). The SB cell line originated from a patient with acute lymphoblastic leukemia and is positive for EBV (CCL-120, ATCC). The Daudi cell line was isolated from a patient with Burkitt's lymphoma (CCL-213, ATCC). Neomycin resistant CD80-expressing Chinese hamster ovary cells (CHO) were generated using IDEC Pharmaceuticals proprietary vector system.

Antibodies

IDEC-114 is a PRIMATIZED® anti-human CD80 mAb that contains human gamma 1 heavy chain (Lot 114S004F, code 3002G710; Lot ZPPB-01) and rituximab is an anti-human CD20 specific mouse-human gamma 1 chimeric antibody (Lot E9107A1; Lot D9097A1). Other antibodies used include the murine anti-human CD80 mAb L307.4 (BD Pharmingen, San Diego, Calif.), the primatized anti-human CD4 mAb CE9.1, with human gamma 1 chain (Lot M2CD4156), and the murine isotype-matched (IgG1) control antibody 3C9 developed at IDEC Pharmaceuticals.

Antibody Binding

Binding of antibodies to CD20 and CD80 molecules expressed on different B-lymphoma lines was determined by flow cytometry. Varying concentrations of test or control antibodies diluted in cold fluorescence-activated cell sorting (FACS) binding buffer was incubated in a cell-culture tube with 1×10⁶ cells to a final volume of 200 μl. IDEC-114 and rituximab were used as test antibodies and CE9.1 was used as the isotype-matched negative control. The cells were incubated for 60 minutes on ice and washed once in FACS wash buffer following incubation. Cells were resuspended in 200 μl of FACS binding buffer, and 2 μl of FITC-conjugated goat F(ab′)₂ anti-human Ig gamma chain specific antibodies (Southern Biotechnology, Birmingham, Ala.) per 10⁶ cells was added. Following further incubation of 30 minutes on ice, cells were washed once and resuspended in 200 μl cold HBSS, and fixed with 200 μl of 1% formaldehyde. The cells were acquired and analyzed by FACSscan and WinList as described above.

Antibody-Dependent Cellular Cytotoxicity (ADCC)

In the ADCC assay, SKW or SB cells and activated human peripheral monocytes (PBMC) were used as targets and effector cells, respectively. PBMC were isolated from whole blood of healthy donors using Histopaque (Sigma-Aldrich Corp., St. Louis, Mo.). The PBMC were cultured at a concentration of 5×10⁶ cells/ml in complete medium with 20 U/ml recombinant human IL-2 (Invitrogen, Carlsbad, Calif.) in 75 cm² tissue culture flasks at 37° C. and 5% CO₂. After overnight culture, 1×10⁶ SKW or SB target cells were labeled with 150 μCi of ⁵¹Cr (Amersham Pharmacia Biotech, Piscataway, N.J.) for 1 hour at 37° C. and 5% CO₂. The cells were washed four times and resuspended in 5 ml of complete medium; 50 μl of cell suspension was dispensed into each well containing equal volume of test or control antibodies. Rituximab (Lot E9107A1) or IDEC-114 (Lot 114S004F, code 3002G710) were used as test antibodies. Isotype matched CE9.1 (Lot M2CD4156) or L307.4 (BD Pharmingen), or a murine isotype-matched (IgG₁) antibody of irrelevant specificity, 3C9, were used. All wells were plated in triplicate into a 96 well, round bottom tissue culture plate. The effector cells were harvested, washed once with complete medium, and added at 1×10⁶ cells in 100 μl volume per well to obtain a 50:1 effector to target ratio. The following control wells were also included in triplicate: target cell incubated with 100 μl complete medium to determine spontaneous release and target cell incubated with 100 μl 0.5% Triton X-100 (Sigma-Aldrich Corp.) to determine maximum release. The culture was incubated for 4 hours at 37° C. and 5% CO₂ and the ⁵¹Cr released in the culture supernatant due to cell lysis was determined by a gamma counter (ISODATA). The cytotoxicity was expressed as the percentage of specific lysis and calculated as follows:

$$- \frac{\begin{matrix}{}^{51} Cr release of test samples - \\ spontaneous {}^{51} Cr release\end{matrix}}{\begin{matrix}Maximum {}^{51} Cr release - \\ spontaneous {}^{51} Cr release\end{matrix}} 100$$

Complement-Dependent Cytotoxicity (CDC)

The CDC activity of IDEC-114 and rituximab was determined using B-cell lines and human complement (C). Dilutions of antibodies were made at 4×concentration and 50 μl was dispensed into each 96 well in triplicates. The SKW or Daudi cells were labeled with ⁵¹Cr (150 μCi/10⁶ cells) for 1 hour at 37° C. and 5% CO₂. The cells were washed four times and resuspended in complete medium, and 1×10⁴ cells in 50 μl were dispensed into each well. One hundred μl of normal human serum complement (Quidel, San Diego, Calif.) diluted 1:4 or 1:8 in complete medium was added. Methods for the spontaneous and maximum release control and set up are described in Section 2.D. (Antibody-Dependent Cellular Cytotoxicity [ADCC]). The cultures were incubated 4 hours at 37° C. and 5% CO₂. The radioactivity released into the culture supernatant was determined by a gamma counter. The formula for calculating the percentage of specific cell lysis is described in Section 0. (Antibody-Dependent Cellular Cytotoxicity [ADCC]).

Tumor Model

A human lymphoma tumor model in severe immunodeficiency (SCID) mice was developed. Briefly, 3×10⁶ to 4×10⁶ SKW cells were intravenously injected into 6- to 8-week old female SCID mice and their survival was monitored for 45 to 60 days. All mice developed a paralytic form of the disease before circumventing to death. Mice that developed severe paralysis were sacrificed and scored as dead. Kaplan-Meier analysis was performed using the Statistical Analysis System (SAS) and p-values were generated by the Log-rank test.

### Example 16

CD80 Expression in Lymphoma

CD80 is transiently expressed on the surface of activated B cells and activated APCs, but is weakly expressed or not expressed on resting B-cells and resting APCs. Since CD80 is a B-cell activation marker, it is expressed primarily on the dividing and/or activated lymphoma cells. Reports suggest that CD80 is constitutively expressed on malignant B cells. To confirm these reports, we tested the expression of CD80 by flow cytometry in a panel of lymphoma and leukemia specimens obtained from 20 patients. Results indicate that CD80 is expressed in lymphomas and leukemias at different densities (Table 3). The highest CD80 expression was observed in follicular, small-cleaved, low-grade lymphoma and in small, non-cleaved Burkitt's lymphoma. The lowest expression was seen in one chronic lymphocytic lymphoma (CLL) sample and in one small, non-cleaved, high-grade lymphoma. CD80 expression on follicular, small-cleaved, low-grade lymphoma samples is presented in Table 4. The CD80 expression on these lymphoma cells ranged from 25% to 90% of the tumor cells in the samples. It is interesting, however, that within the same lymphoma the “large” cells were 90% to 100% positive, while the “small” cells were 25% to 100% positive. It is possible that CD80 is expressed on proliferating or activated malignant B cells, which may account for the variability of expression within lymphoma samples tested.

TABLE 3
Expression of CD80 on Lymphoma/Leukemia Specimens

| | Positive | |
|---|---|---|
| Lymphoma | Specimens* | Expression Level^{†} |
| Follicular, small-cleaved, low-grade | 12/12 | Medium to High |
| lymphoma | | |
| Follicular, large-cell, intermediate-grade | 1/1 | Low |
| lymphoma | | |
| Small, non-cleaved Burkitt's lymphoma | 2/2 | High |
| Small, non-cleaved, high-grade | 1/1 | Weak |
| lymphoma | | |
| Chronic lymphocytic leukemia (CLL)/ | 2/2 | Weak to Medium |
| small lymphocytic lymphoma (SLL) | | |
| Mantle cell lymphoma (CLL variant) | 2/2 | Medium |

*Positive samples/samples tested
^{†}Expression level is a subjective value estimated by analysis of flow cytometry data, and is the percentage of positive gated cells over the control antibody; weak = <10%, low = 10% to 25%, medium = 25% to 50%, and high = <50%

TABLE 4
Expression of CD80 on Follicular, Small-Cleaved,
Low-Grade Lymphoma Specimens

| Sample | Percentage of CD80 Expression |
|---|---|
| 1 | 79% |
| 2 | 25% |
| 3 | 26% (Small) |
| | 89% (Large) |
| 4 | 99% |
| 5 | 100% (Small) |
| | 99% (Large) |
| 6 | 67% (Small) |
| | 95% (Large) |
| 7 | 67% (Small) |
| | 89% (Large) |
| 8 | 64% (Small) |
| 9 | 100% |
| 10 | 70% |
| 11 | 88% |
| 12 | 84% |

### Example 17

Binding Activity of IDEC-114

The binding activity of IDEC-114 to CD80 expressed on CD80-CHO transfectant and on lymphoma cell lines was determined by flow cytometry shows the specific binding of IDEC-114 from two different lots (Lot 114S004F and Lot 114S015) to CD80-CHO cells in a concentration dependent fashion. As expected, isotype-matched control antibody of irrelevant specificity (IDEC-152) did not bind to CD80-CHO cells. Testing of IDEC-114 for binding to CD80 on SKW and SB lymphoma cell lines showed a lower binding than that of rituximab as demonstrated by a lower percentage of positive cells (Table 5) and lower mean fluorescence intensity (Table 6).

TABLE 5
Binding of Antibodies to B-Lymphoma Cell Lines

| Antibody | Intensity of Binding Activity* | | |
|---|---|---|---|
| (10 μg/ml) | SKW | SB | Daudi |
| IDEC-114 | 1.8 | 2 | 3 |
| Rituximab | 20 | 52 | 60 |
| CE9.1 (control mAb) | 1 | 1 | 1 |

*Binding to cells was determined by flow cyometry at saturating concentrations of antibody. Intensity of Binding Activity = MFI of test antibody ÷ MFI of control antibody.

TABLE 6
Relative CD80 Antigen Density on CD80-CHO and SB Cells

| Cell | MFI* |
|---|---|
| CD80-CHO | 676 |
| SB (B-lymphoma line) | 189 |
| PBMC Control | 51 |
| PBMC Activated | 44 |

*The relative CD80 antigen was measured by mean fluorescence intensity (MFI). Values are expressed in units after subtraction of background intrinsic fluorescence.

### Example 18

Antibody-Dependent Cellular Cytotoxicity (ADCC)

The ability of IDEC-114 to mediate ADCC of B-cell lymphoma was determined. FIG. 11 shows the ADCC activity of IDEC-114 and rituximab on CD20⁺/CD80⁺ SB and SKW cells. Overall, higher levels of ADCC activity were observed with SB cells than with SKW cells. IDEC-114 showed a dose-dependent killing of SB and SKW cells with a maximum killing of 75% and 46%, respectively, at 10 μg/ml. Rituximab at comparable antibody concentrations showed higher ADCC activity (97% on SB cells and 65% on SKW cells) than IDEC-114, which correlated with higher cell-binding activity of rituximab compared with IDEC-114. As expected, murine L307.4, which does not bind to the human Fc receptor, showed weak ADCC activity. Only background levels of ADCC were observed with isotype human and murine controls (CE9.1 and 3C9, respectively).

Experiments were performed to determine the effect of combining IDEC-114 with rituximab to increase host effector mediated killing of tumor cells. In these experiments, a fixed concentration of IDEC-114 was combined with varying concentrations of rituximab to reflect a scenario where low CD20 density with normal B7 expression on B-lymphoma cells could lead to effective tumor killing. The results obtained in FIG. 12 show that the combination of IDEC-114 with rituximab leads to enhanced ADCC activity on SKW lymphoma cells. IDEC-114 at a fixed concentration of 10 μg/ml in combination with rituximab concentrations of 0.1 to 0.01 μg/ml mediated an enhanced killing of SKW cells. The results obtained using host effector cells from two donors showed the same trend in ADCC activity.

### Example 19

Complement-Dependent Cytotoxicity (CDC)

Activation of the complement cascade following binding of rituximab to the CD20 antigen results in efficient killing of B-lymphoma cells in vitro. Therefore, we evaluated the capacity of IDEC-114 to mediate complement-dependent killing of CD80⁺ target cells. Results showed that IDEC-114 mediates CDC of CD80-expressing CHO cells (a). However, binding of IDEC-114 to CD80⁺ Daudi and SKW lymphoma cell lines showed no evidence of CDC (b) and (FIG. 14a) (c), respectively. In contrast, rituximab showed CDC activity on both cell lines, although the Daudi cell line was more sensitive to CDC than the SKW cell line (FIG. 14b) and (c) respectively). The lack of CDC activity observed with IDEC-114 on lymphoma cell lines may be due to lower CD80 expression than CD20 expression on these cell lines (Table 5 and Table 6). Alternatively, lymphoma cells have been reported to be resistant to complement-mediated killing. Resistance was associated with the cell-surface expression of C-regulatory proteins such as CD55 and CD59 antigens. Flow cytometric analysis demonstrated higher expression of CD55 on SKW cells compared with Daudi cells (results not shown), which correlated with lower cytolytic activity of rituximab on SKW cells. The lack of CDC activity by IDEC-114 observed on both SKW and Daudi cells was not related to higher levels of CD55 expression since rituximab exhibited CDC on both CD55^{low} Daudi cells and CD55^{high} SKW cells (FIG. 14b and FIG. c respectively).

### Example 20

In Vivo Therapeutic Effect of IDEC-114 and Rituximab Single-Agent Therapies in Lymphoma

Human SKW lymphoma cells were inoculated intravenously (IV) into BALB/c SCID mice. After inoculation, SKW cells disseminate throughout the mouse and grow primarily in the lungs and liver. Treatment with IDEC-114 or rituximab was initiated 1 day after tumor inoculation and repeated every 2 days for six total injections. FIG. 15 shows the antitumor response of single-agent IDEC-114 and single-agent rituximab therapy using three doses (100, 200, and 400 μg) of the antibody. IDEC-114 and rituximab single-agent therapy showed inhibition of disease progression at all doses. The antitumor response observed with IDEC-114 was comparable to the antitumor response of rituximab at the same dose and treatment schedule.

### Example 21

In Vivo Therapeutic Effect of IDEC-114 Rituximab Combination Therapy in Lymphoma

Based on the antitumor activity of IDEC-114 as a single agent, a combination of IDEC-114 and rituximab was evaluated in the same tumor model at the same dosing schedule described in previously. (In Vivo Therapeutic Effect of IDEC-114 and Rituximab Single-Agent Therapies in Lymphoma). SKW/SCID mice were injected with 200 μg of IDEC-114 and 200 μg of rituximab, and compared with the mice injected with either 200 μg or 400 μg of IDEC-114 or 200 μg or 400 μg of rituximab. FIG. 16 shows the survival advantage of mice treated with a IDEC-114 rituximab combination therapy compared with mice treated with either IDEC-114 or rituximab as a single-agent therapy. Results show that the combination of IDEC-114 and rituximab leads to increased disease-free survival compared with either antibody alone. In the combination therapy group, 70% (7/10) of the mice survived for more than 50 days after the last antibody injection. In contrast, less than 10% of mice treated with IDEC-114 or rituximab alone were alive at the end of the study. Survival data were analyzed by Kaplan-Meier and Log-rank tests (Table 7). The IDEC-114 rituximab combination therapy produced a statistically greater response than 200 μg or 400 μg of IDEC-114 or rituximab single-agent therapy.

TABLE 7
Comparison of IDEC-114/Rituximab Combination Therapy with
IDEC-114 or Rituximab Single-Agent Therapy

| Comparison | p-value* |
|---|---|
| IDEC-114/rituximab combination vs. saline control group | 0.0001 |
| IDEC-114/rituximab combination vs. rituximab | 0.0008 |
| (200 μg and 400 μg) | |
| IDEC-114/rituximab combination vs. IDEC-114 | 0.0017 |
| (200 μg and 400 μg) | |
| IDEC-114/rituximab combination vs. IDEC-114 (200 μg) | 0.0403 |
| IDEC-114/rituximab combination vs. IDEC-114 (400 μg) | 0.001 |

*p-value generated by Log-rank test

### Example 22

Combination Treatment of IDEC-114 With Adriamycin Provide Increased Disease-free Survival in Human Lymphoma-muse Model

Human SKW lymphoma cells were inoculated intravenously (IV) into BALB/c SCID mice. After inoculation, SKW cells disseminate throughout the mouse and grow primarily in the lungs and liver. Treatment with IDEC-114 was initiated 1 day after tumor inoculation and repeated every 2 days for six total injections. Treatment with Aadriamycin (ADM) was initiated on day 3 and repeated every week for 3 total injections. FIG. 19 shows the anti-tumor response of single-agent IDEC-114 at 100 ug dose and ADM at 1.25 mg/Kg and 2.5 mg/Kg and the combination of IDEC-114 at 100 ug with ADM at 1.25 mg/Kg or 2.5 mg/Kg. At 100 ug dose IDEC-114 as single-agent therapy showed delay in disease progression with 50% survival at day 38, whereas, ADM at 2.5 mg/Kg dose was effective with 50% survival at day 41. No anti-tumor effect was observed with ADM at 1.25 mg/Kg dose. The anti-tumor response observed with IDEC-114 was comparable to the anti-tumor response of ADM at 2.5 mg/Kg. In addition to the antitumor activity of IDEC-114 and ADM as a single agent, a combination of IDEC-114 and ADM was evaluated in the same tumor model at the same dosing schedule. Mice were injected with combination of 100 μg of IDEC-114 and ADM at 1.25 mg/Kg or 2.5 mg/Kg doses. Results show that the combination of IDEC-114 and with 2.5 mg/Kg leads to increased disease-free survival compared with either IDEC-114 or 2.5 mg/Kg ADM alone. In the combination therapy group, 77% (7/9) of the mice survived at the end of the study. In contrast, less than 25% of mice treated with IDEC-114 or ADM alone were alive at the end of the study. Combination of 100 ug dose of IDEC-114 with 1.25 mg/Kg ADM did not show increased in survival. The IDEC-114/ADM (2.5 mg/kg) combination therapy produced a statistically greater response than 100 ug of IDEC-114 or 2.5 mg/Kg ADM single-agent therapy.

### CONCLUSION

The expression of CD80 on malignant B cells and the results herein indicate CD80 can be used as a target for antibody therapy of lymphoma. In addition, antibodies against CD80 have been shown to synergize with rituximab by increasing the antibody density on the lymphoma cells rendering them more susceptible to killing mechanisms such as ADCC. In a disseminated human B lymphoma/SCID mouse model, IDEC-114 as a single-agent therapy showed antitumor activity comparable to that of rituximab. In the same model, the combination therapy of IDEC-114 and rituximab showed a synergistic antitumor response, which was significantly higher than IDEC-114 or rituximab single-agent therapy. Experimental evidence suggests that the primary mechanism for the observed antitumor response is Fc-dependent host effector cell mediated cytotoxicity. Also, combination treatment of IDEC-114 and a chemotherapeutic agent was shown to provide for enhanced survival in a SCID mouse animal model for lymphoma. The following references are cited in this application and are incorporated by reference in their entirety herein.

### REFERENCES

1. Freeman G, Freedman A, Segil J, Lee G, Whitman J, Nadler L. B7, A new member of the Ig superfamily with unique expression on activated and neoplastic B cells. J Immunol 1989;143:2714-22.

2. Razi-Wolf Z, Freeman G, Galvin F, Benacerraf B, Nadler L, Reiser H. Expression and function of the murine B7 antigen, the major costimulatory molecule expressed by peritoneal exudate cells. Proc Natl Acad Sci USA 1992;89:4210-4.

3. Azuma M, Yssel H, Phillips J, Spits H, Lanier L. Functional expression of B7/BB1 on activated T lymphocytes. J Exp Med 1993;177:845-50.

4. Hathcock KS, Laszlo G, Pucillo C, Linsley P, Hodes RJ. Comparative analysis of B7-1 and B7-2 costimulatory ligands: expression and function. J Exp Med 1994;180(2):631-40.

5. Anderson DE, Sharpe AH, Hafler DA. The B7-CD28/CTLA-4 costimulatory pathways in autoimmune disease of the central nervous system. Curr Opin Immunol 1999;11(6):677-83.

6. Dorfman DM, Schultze JL, Shahsafaei A, Michalak S, Gribben JG, Freeman GJ, et al. In vivo expression of B7-1 and B7-2 by follicular lymphoma cells can prevent induction of T-cell anergy but is insufficient to induce significant T-cell proliferation. Blood 1997;90(11):4297-306.

7. Rituxan® (rituximab). Package Insert. San Diego: IDEC Pharmaceuticals Corporation; 4-19, 2001, 2.

8. Reff ME, Carner K, Chambers KS, Chinn PC, Leonard JE, Raab R, et al. Depletion of B cells in vivo by a chimeric mouse human monoclonal antibody to CD20. Blood 1994;83(2):435-45.

1. A method for potentiating the ADCC and/or CDC activity of an anti-CD80 (B7.1) antibody against CD80 positive cells by administering said anti-CD80 antibody in combination with an anti-CD20 antibody.

2. The method of claim 1 wherein said antibodies are administered in either order or together.

3. The method of claim 2 wherein the anti-CD20 antibody is administered first.

4. The method of claim 2 wherein the anti-CD80 antibody is administered first.

5. The method of claim 1 wherein the anti-CD80 antibody is a human, humanized or chimeric antibody containing human constant regions.

6. The method of claim 5 wherein said anti-CD80 antibody contains human IgG₁ or IgG₃ constant regions.

7. The method of claim 1 wherein the anti-CD20 antibody is Rituxan®.

8. The method of claim 1 wherein the anti-CD80 antibody is IDEC-114.

9. A method of treating B cell malignancy comprising administering a therapeutically effective amount of an anti-CD80 antibody.

10. The method of claim 9 wherein said antibody is a human, humanized or chimeric antibody containing human constant regions.

11. The method of claim 10 wherein said constant regions are human IgG₁ or IgG₃ constant regions.

12. The method of claim 9 wherein said antibody binds the same epitope as IDEC-114 or competes with IDEC-114 for binding to CD80.

13. The method of claim 9 wherein said antibody exhibits ADCC and/or CDC activity against B cell lymphoma cells.

14. The method of claim 9 wherein said antibody is IDEC-114.

15. The method of claim 9 wherein said B cell malignancy is a B cell lymphoma or leukemia.

16. The method of claim 15 wherein said B cell malignancy is a B cell lymphoma.

17. The method of claim 16 wherein said B cell lymphoma is selected from the group consisting of Hodgkin's lymphoma, non-Hodgkin's lymphoma, low grade/follicular non-Hodgkin's lymphoma (NHL), cell lymphoma (FCC), mantle cell lymphoma (MCL), diffuse large cell lymphoma (DLCL), small lymphocyte (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia.

18. The method of claim 16 wherein said leukemia is ALL-L3 (Burkitt's type leukemia), chronic lymphocytic leukemia (CLL) and monocytic cell leukemia.

19. The method of claim 17 wherein said lymphoma is a non-Hodgkin's lymphoma.

20. A combination therapy for the treatment of a B cell malignancy comprising administering a therapeutically effective amount of an anti-CD80 antibody and an anti-CD20 antibody.

21. The method of claim 1 wherein said antibodies are administered in either order or together.

22. The method of claim 20 wherein the anti-CD20 antibody is administered first.

23. The method of claim 20 wherein the anti-CD80 antibody is administered first.

24. The method of claim 20 wherein in anti-CD80 antibody is a human, humanized or chimeric antibody containing human constant regions.

25. The method of claim 24 wherein said anti-CD80 antibody contains human IgG₁ or IgG₃ constant regions.

26. The method of claim 20 wherein the anti-CD80 antibody is IDEC-114.

27. The method of claim 20 wherein said B cell malignancy is a B cell lymphoma or leukemia.

28. The method of claim 27 wherein said B cell malignancy is a B cell lymphoma.

29. The method of claim 27 wherein said B cell lymphoma is selected from the group consisting of Hodgkin's lymphoma, non-Hodgkin's lymphoma, low grade/follicular non-Hodgkin's lymphoma (NHL), cell lymphoma (FCC), mantle cell lymphoma (MCL), diffuse large cell lymphoma (DLCL), small lymphocyte (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia.

30. The method of claim 27 wherein said leukemia is ALL-L3 (Burkitt's type leukemia), chronic lymphocytic leukemia (CLL) or monocytic cell leukemia.

31. The method of claim 28 wherein said lymphoma is a non-Hodgkin's lymphoma.

32. A combination therapy for treatment of a B cell malignancy comprising administering a synergistically effective amount of an anti-CD80 antibody and at least one chemotherapeutic agent.

33. The therapy of claim 32 wherein said antibody and chemotherapeutic are administered in either order or together.

34. The method of claim 32 wherein the antibody is administered first.

35. the method of claim 32 wherein the chemotherapeutic is administered first.

36. The method of claim 32 wherein in anti-CD80 antibody is a human, humanized or chimeric antibody containing human constant regions.

37. The method of claim 36 wherein said anti-CD80 antibody contains human IgG₁ or IgG₃ constant regions.

38. The method of claim 37 wherein the anti-CD80 antibody is IDEC-114.

39. The method of claim 32 wherein said B cell malignancy is B cell lymphoma or leukemia.

40. The method of claim 32 wherein said B cell malignancy is a B cell lymphoma.

41. The method of claim 40 wherein said B cell lymphoma is selected from the group consisting of Hodgkin's lymphoma, non-Hodgkin's lymphoma, low grade/follicular non-Hodgkin's lymphoma (NHL), cell lymphoma (FCC), mantle cell lymphoma (MCL), diffuse large cell lymphoma (DLCL), small lymphocyte (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia.

42. The method of claim 39 wherein said leukemia is selected from ALL-L3 (Burkitt's type leukemia), chronic lymphocytic leukemia (CLL) and monocytic cell leukemia.

43. The method of claim 39 wherein said lymphoma is a non-Hodgkin's lymphoma.

44. The method of claim any one of claims 1-43 wherein the administered antibody or antibodies are radiolabeled.

45. The method of claim 44 wherein said radiolabel is ¹³¹I or ⁹⁰Y.

46. The method of anyone of claims 1-43 wherein the administered antibody or antibodies are administered by a means selected from the group consisting of parenterally, orally, intraartiniolly, intrapertineolly, intramuscularly, rectally, vaginally, and subcutaneously.

47. The method of claim 46 wherein the administered antibody or antibodies comprise a reconstituted lyophilised formulation.

48. The method of claim 47 wherein said administered formulation comprises a lyoprotectant.

49. The method of claim 48 wherein said lyoprotectant is a polyol, sucrose or trelalose.

50. The method of any one of claims 1-43 wherein the antibody dosage ranges from 0.05 to 100 mg/kg body weight.

51. The method of claim 50 wherein said dosage ranges from 0.5 to 10 mg/kg body weight.


SEQUENCE LISTING (12 sequences)

SEQ ID NO: 1 - DNA, 705 bp, Artificial Sequence
  Description of Artificial Sequence Synthetic primatized nucleotide sequence
atgagggtccccgctcagctcctggggctcctgctgctctggctcccaggtgcacgatgtgcctatgaactgactcagccaccctcggtgtcagtgtccccaggacagacggccaggatcacctgtgggggagacaacagtagaaatgaatatgtccactggtaccagcagaagccagcgcgggcccctatactggtcatctatgatgatagtgaccggccctcagggatccctgagcgattctctggctccaaatcagggaacaccgccaccctgaccatcaacggggtcgaggccggggatgaggctgactattactgtcaggtgtgggacagggctagtgatcatccggtcttcggaggagggacccgggtgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatga

SEQ ID NO: 2 - protein, 234 aa, Artificial Sequence
  Description of Artificial Sequence Synthetic primatized peptide sequence
MRVPAQLLGLLLLWLPGARCAYELTQPPSVSVSPGQTARITCGGDNSRNEYVHWYQQKPARAPILVIYDDSDRPSGIPERFSGSKSGNTATLTINGVEAGDEADYYCQVWDRASDHPVFGGGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - DNA, 1431 bp, Artificial Sequence
  Description of Artificial Sequence Synthetic primatized nucleotide sequence
atgaaacacctgtggttcttcctcctcctggtggcagctcccagatgggtcctgtcccaggtgaagctgcagcagtggggcgaaggacttctgcagccttcggagaccctgtcccgcacctgcgttgtctctggtggctccatcagcggttactactactggacctggatccgccagaccccagggaggggactggagtggattggccatatttatggtaatggtgcgaccaccaactacaatccctccctcaagagtcgagtcaccatttcaaaagacacgtccaagaaccagttcttcctgaacttgaattctgtgaccgacgcggacacggccgtctattactgtgcgagaggccctcgccctgattgcacaaccatttgttatggcggctgggtcgatgtctggggcccgggagacctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 476 aa, Artificial Sequence
  Description of Artificial Sequence Synthetic primatized peptide sequence
MKHLWFFLLLVAAPRWVLSQVKLQQWGEGLLQPSETLSRTCVVSGGSISGYYYWTWIRQTPGRGLEWIGHIYGNGATTNYNPSLKSRVTISKDTSKNQFFLNLNSVTDADTAVYYCARGPRPDCTTICYGGWVDVWGPGDLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 720 bp, Artificial Sequence
  Description of Artificial Sequence Synthetic primatized nucleotide sequence
atgagcctccctgctcagctcctcgggctgctattgctctgcgtccccgggtccagtggggaagttgtgatgactcagtctccactgtcccttcccatcacacctggagagccggcctccatctcctgtaggtctagtcaaagccttaaacacagtaatggagacaccttcctgagttggtatcagcagaagccaggccaacctccaaggctcctgatttataaggtttctaaccgggactctggggtcccagacagattcagcggcagtggggcagggacagatttcacactgaaaatcagcgcagtggaggctgaagatgttggggtttatttctgcgggcaaggtacaaggactcctcccactttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 6 - protein, 239 aa, Artificial Sequence
  Description of Artificial Sequence Synthetic primatized peptide sequence
MSLPAQLLGLLLLCVPGSSGEVVMTQSPLSLPITPGEPASISCRSSQSLKHSNGDTFLSWYQQKPGQPPRLLIYKVSNRDSGVPDRFSGSGAGTDFTLKISAVEAEDVGVYFCGQGTRTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 1437 bp, Artificial Sequence
  Description of Artificial Sequence Synthetic primatized nucleotide sequence
atgggttggagcctcatcttgctcttccttgtcgctgttgctacgcgtgtccagtgtgaggtgcaactggtggagtctgggggaggcttggtccagcctggcgggtccctgagagtctcctgtgcagtctctggattcaccttcagtgaccactacatgtattggttccgccaggctccagggaaggggccggaatgggtaggtttcattagaaacaaaccgaacggtgggacaacagaatacgccgcgtctgtgaaagacagattcaccatctccagagatgattccaaaagcatcgcctatctgcaaatgagcagcctgaaaatcgaggacacggccgtctattactgtactacatcctacatttcacattgtcggggtggtgtctgctatggaggttacttcgaattctggggccagggcgccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - protein, 478 aa, Artificial Sequence
  Description of Artificial Sequence Synthetic primatized peptide sequence
MGWSLILLFLVAVATRVQCEVQLVESGGGLVQPGGSLRVSCAVSGFTFSDHYMYWFRQAPGKGPEWVGFIRNKPNGGTTEYAASVKDRFTISRDDSKSIAYLQMSSLKIEDTAVYYCTTSYISHCRGGVCYGGYFEFWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 711 bp, Artificial Sequence
  Description of Artificial Sequence Synthetic primatized nucleotide sequence
atgagggtccccgctcagctcctggggctcctgctgctctggctcccaggtgcacgatgtgagtctgtcctgacacagccgccctcagtgtctggggccccagggcagaaggtcaccatctcgtgcactgggagcacctccaacattggaggttatgatctacattggtaccagcagctcccaggaacggcccccaaactcctcatctatgacattaacaagcgaccctcaggaatttctgaccgattctctggctccaagtctggtaccgcggcctccctggccatcactgggctccagactgaggatgaggctgattattactgccagtcctatgacagcagcctgaatgctcaggtattcggaggagggacccggctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatga

SEQ ID NO: 10 - protein, 236 aa, Artificial Sequence
  Description of Artificial Sequence Synthetic primatized peptide sequence
MRVPAQLLGLLLLWLPGARCESVLTQPPSVSGAPGQKVTISCTGSTSNIGGYDLHWYQQLPGTAPKLLIYDINKRPSGISDRFSGSKSGTAASLAITGLQTEDEADYYCQSYDSSLNAQVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - DNA, 1431 bp, Artificial Sequence
  Description of Artificial Sequence Synthetic primatized nucleotide sequence
atgaaacacctgtggttcttcctcctcctggtggcagctcccagatgggtcctgtcccaggtgcagctgcaggagtcgggcccaggactggtgaagccttcggagaccctgtccctcacctgcgctgtctctggtggctccatcagcggtggttatggctggggctggatccgccagcccccagggaaggggctggagtggattgggagtttctatagtagtagtgggaacacctactacaacccctccctcaagagtcaagtcaccatttcaacagacacgtccaagaaccagttctccctgaagctgaactctatgaccgccgcggacacggccgtgtattactgtgtgagagatcgtcttttttcagttgttggaatggtttacaacaactggttcgatgtctggggcccgggagtcctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 476 aa, Artificial Sequence
  Description of Artificial Sequence Synthetic primatized peptide sequence
MKHLWFFLLLVAAPRWVLSQVQLQESGPGLVKPSETLSLTCAVSGGSISGGYGWGWIRQPPGKGLEWIGSFYSSSGNTYYNPSLKSQVTISTDTSKNQFSLKLNSMTAADTAVYYCVRDRLFSVVGMVYNNWFDVWGPGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK